(19) United States
(12) Patent Application Publication
(10) Pub. No.: US 2015/0246118 A1
(43) Pub. Date: Sep. 3, 2015

(54) LYVE-1 ANTAGONISTS FOR PREVENTING OR TREATING A PATHOLOGICAL CONDITION ASSOCIATED WITH LYMPHANGIOGENESIS

(71) Applicants: INSERM (INSTITUT NATIONAL DE LA SANTÉ ET DE LA RECHERCHE MÉDICALE), Paris (FR); UNIVERSITÉ DE BORDEAUX I, Talence (FR)

(72) Inventor: Andreas Bikfalvi, Talence (FR)

(21) Appl. No.: 14/438,465

(22) PCT Filed: Oct. 24, 2013
(86) PCT No.: PCT/EP2013/072296

§ 371 Date: Apr. 24, 2015

(30) Foreign Application Priority Data
Oct. 26, 2012 (EP) 12306330.7

(51) Int. Cl. A61K 39/395; C07K 16/28; G01N 33/50; A61K 31/713; A61K 45/06; C07K 16/30; A61K 38/17; C12N 15/113

(52) U.S. Cl. CPC A61K 39/3955; A61K 38/177; C07K 16/28; C12N 15/113; A61K 31/713; A61K 45/06; A61K 39/39558; C07K 16/30; G01N 33/502; G01N 33/5011; C07K 2317/76; C12N 2320/30; C12N 2310/14; G01N 2333/705; G01N 2333/503; G01N 2800/245; G01N 2800/24

(57) ABSTRACT

The present invention relates to the prevention or treatment of pathological conditions associated with lymphoangiogenesis (e.g. cancer and eye diseases). The present invention also relates to a method for screening for screening a compound capable of reducing or inhibiting lymphangiogenesis and which may be useful for preventing or treating a pathological condition associated with lymphangiogenesis.

### FIELD OF THE INVENTION

The invention relates to methods for preventing or treating a pathological condition associated with lymphoangiogenesis (e.g. cancer and eye diseases). The invention also relates to methods for inhibiting or preventing tumor metastasis and tumor lymphoangiogenesis.

### BACKGROUND OF THE INVENTION

The lymphatic system is important to maintain fluid homeostasis by collecting fluid that leaks from capillary blood vessels and returning it to the blood circulation⁽¹⁾. Perturbations in the development, maintenance and function of the lymphatic system can lead to a variety of pathological lymphatic disorders including lymphedema, inflammation and tumor metastasis⁽²⁾. Tumor metastasis the spread of cancer cells to distant organs, is the main cause of death for cancer patients. Tumor metastasis is often mediated by lymphatic vessels that invade the primary tumor, and an early sign of metastasis is the presence of cancer cells in the regional lymph node (the first lymph node colonized by metastasizing cancer cells from a primary tumor). Understanding the interplay between tumorigenesis and lymphangiogenesis (the formation of lymphatic vessels associated with tumor growth) have provides new insights into mechanisms that modulate metastatic spread. Thus, the understanding of the molecular and cellular regulation of lymphangiogenesis has greatly advanced in recent years with the identification of the lymphangiogenic vascular endothelial growth factors VEGF-C and VEGF-D and their lymphatic vessel-specific receptor VEGF receptor-3 (VEGFR-3)⁽³⁾.

These insights have helped to define new molecular targets that could be used to block lymphatic vessel-mediated metastasis and increase patient survival. For instance, anti-VEGF-C antibodies (e.g. VGX-100), anti-VEGFR-3 antibodies (e.g. IMC-035) or recombinant soluble VEGFR-3 (e.g. VGX-300) are now developed and are currently in clinical trials.

Moreover, the repertoire of lymphangiogenic factors has been increased when it became apparent that other growth factor molecules or angiogenic factors were also regulating lymphangiogenesis⁽⁴⁻⁶⁾. As such, it has been recognized that Fibroblast Growth Factor-2 (FGF2) induces lymphangiogenesis by both direct and indirect mechanisms; it binds lymphatic endothelial cells (LEC) and stimulates their proliferation and migration in vitro^{(7,8)}; in addition recent findings suggested that FGF2 reciprocally interacts with VEGF-C leading to additive lymphangiogenic activity and metastasis^{(9,10)}.

Hence, it is highly desirable to have relevant means of modulating the FGF2-induced lymphangiogenesis (or at least one or more of the biological effects induced by FGF2 such as stimulation of lymphatic endothelial migration, proliferation and tubulogenesis). Accordingly, identifying new molecular targets that could be used to inhibit tumor lymphangiogenesis and hence lymphatic vessel-mediated metastasis is needed to increase patient survival. However, it remains unknown which receptor mediates the FGF2-induced lymphangiogenesis.

### SUMMARY OF THE INVENTION

In a first aspect, the present invention relates to a Lymphatic Vessel Endothelial Hyaluronan Receptor-1 (LYVE-1) antagonist for use in the prevention or the treatment of a pathological condition associated with lymphangiogenesis.

In a second aspect, the present invention also relates to a LYVE-1 antagonist for use in the prevention of tumor metastasis.

In a third aspect, the present invention further relates to a pharmaceutical composition comprising a LYVE-1 antagonist of the invention and a pharmaceutically acceptable carrier.

In another aspect, the present invention relates to a kit-of-part composition comprising at least a LYVE-1 antagonist of the invention and an additional therapeutic agent.

In still another aspect, the present invention relates to a method for screening a LYVE-1 antagonist comprising the steps consisting of:

a) determining the ability of a candidate compound to inhibit the interaction between a LYVE-1 polypeptide and a FGF2 polypeptide, and
b) selecting positively the candidate compound that inhibits said interaction.

### DETAILED DESCRIPTION OF THE INVENTION

The present invention is based on the unexpected discovery that LYVE-1 contributes to FGF's biological activity and signaling in lymphatic endothelial cells. As disclosed herein, FGF2 is able to bind LYVE-1 using AlphaScreen®, or after surface immobilization or in solution. FGF2 binds to LYVE-1 with a higher affinity than any other known LYVE-1-binding molecules such as hyaluronan or PDGF-BB. Glycosylation of LYVE-1 is important for FGF2 binding. Furthermore, FGF2 interacts with LYVE-1 when overexpressed in CHO cells. As shown herein, soluble LYVE-1 and knock-down of LYVE-1 in lymphatic endothelial cells impaired FGF2 signalling and functions. Furthermore, FGF2 but not VEGF-C-induced in vivo lymphangiogenesis, was also inhibited. Conversely, FGF2 also modulates LYVE-1 expression in cells and ex vivo.

Thus, blocking lymphangiogenesis by targeting FGF2 and its receptor LYVE-1 can be effective not only for prevention, but also for treatment of cancer and tumor metastasis as well as of any pathological condition associated with lymphangiogenesis such as eye diseases including corneal transplant rejection.

### DEFINITIONS

Throughout the specification, several terms are employed and are defined in the following paragraphs.

The terms “LYVE-1” or “Lymphatic Vessel Endothelial Hyaluronan Receptor-1” (also known as CRSBP-1), as used herein, refer to any native or variant (whether native or synthetic) LYVE-1 polypeptide. LYVE-1 is well known in the art and refers to a homolog of the hyaluronan receptor CD44, and is one of the most widely used markers of lymphatic endothelial cells in normal and tumor tissues. Thus, the LYVE-1 gene encodes a 322 amino acid polypeptide and includes a type I integral membrane glycoprotein, a transmembrane domain and a cytoplasmic region. The naturally occurring human LYVE-1 gene has a nucleotide sequence as shown in Genbank Accession number NM_{—}006691 and the naturally occurring human LYVE-1 protein has an aminoacid sequence as shown in Genbank Accession number NP_{—}006682 as depicted in SEQ ID NO: 1 (hereafter):

(SEQ ID NO: 1)
MARCFSLVLLLTSIWTTRLLVQGSLRAEELSIQVSCRIMGITLVSKKANQ
QLNFTEAKEACRLLGLSLAGKDQVETALKASFETCSYGWVGDGFVVISRI
SPNPKCGKNGVGVLIWKVPVSRQFAAYCYNSSDTWTNSCIPEIITTKDPI
FNTQTATQTTEFIVSDSTYSVASPYSTIPAPTTTPPAPASTSIPRRKKLI
CVTEVFMETSTMSTETEPFVENKAAFKNEAAGFGGVPTALLVLALLFFGA
AAGLGFCYVKRYVKAFPFTNKNQQKEMIETKVVKEEKANDSNPNEESKKT
DKNPEESKSPSKTTVRCLEAEV.

The term “polypeptide” means herein a polymer of amino acids having no specific length. Thus, peptides, oligopeptides and proteins are included in the definition of “polypeptide” and these terms are used interchangeably throughout the specification, as well as in the claims. The term “polypeptide” does not exclude post-translational modifications that include but are not limited to phosphorylation, acetylation, glycosylation and the like.

By an “isolated” polypeptide, it is intended that the polypeptide is not present within a living organism, e.g. within human body. However, the isolated polypeptide may be part of a composition or a kit. The isolated polypeptide is preferably purified.

A “native sequence” polypeptide refers to a polypeptide having the same amino acid sequence as a polypeptide derived from nature. Thus, a native sequence polypeptide can have the amino acid sequence of naturally-occurring polypeptide from any mammal (including human. Such native sequence polypeptide can be isolated from nature or can be produced by recombinant or synthetic means. The term “native sequence” polypeptide specifically encompasses naturally-occurring truncated or secreted forms of the polypeptide (e. g., an extracellular domain sequence), naturally-occurring variant forms (e. g., alternatively spliced forms) and naturally-occurring allelic variants of the polypeptide.

A polypeptide “variant” refers to a biologically active polypeptide having at least about 80% amino acid sequence identity with the native sequence polypeptide. Such variants include, for instance, polypeptides wherein one or more amino acid residues are added, or deleted, at the N- or C-terminus of the polypeptide. Ordinarily, a variant will have at least about 80% amino acid sequence identity, more preferably at least about 90% amino acid sequence identity, and even more preferably at least about 95% amino acid sequence identity with the native sequence polypeptide.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% (5 of 100) of the amino acid residues in the subject sequence may be inserted, deleted, or substituted with another amino acid.

In the frame of the present application, the percentage of identity is calculated using a global alignment (i.e., the two sequences are compared over their entire length). Methods for comparing the identity and homology of two or more sequences are well known in the art. The “needle” program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is for example available on the ebi.ac.uk world wide web site. The percentage of identity in accordance with the invention is preferably calculated using the EMBOSS::needle (global) program with a “Gap Open” parameter equal to 10.0, a “Gap Extend” parameter equal to 0.5, and a Blosum62 matrix.

Polypeptides consisting of an amino acid sequence “at least 80%, 85%, 90%, 95%, 96%, 97%, 98% or 99% identical” to a reference sequence may comprise mutations such as deletions, insertions and/or substitutions compared to the reference sequence. The polypeptide consisting of an amino acid sequence at least 80%, 85%, 90%, 95%, 96%, 97%, 98% or 99% identical to a reference sequence may correspond to an allelic variant of the reference sequence. It may for example only comprise substitutions compared to the reference sequence. The substitutions preferably correspond to conservative substitutions as indicated in the table below.

| Conservative substitutions | Type of Amino Acid |
|---|---|
| Ala, Val, Leu, lle, Met, | Amino acids with aliphatic hydrophobic |
| Pro, Phe, Trp | side chains |
| Ser, Tyr, Asn, Gln, Cys | Amino acids with uncharged but polar |
| | side chains |
| Asp, Glu | Amino acids with acidic side chains |
| Lys, Arg, His | Amino acids with basic side chains |
| Gly | Neutral side chain |

A polypeptide “fragment”, as used herein, refers to a biologically active polypeptide that is shorter than a reference polypeptide (e.g. a fragment of the extracellular domain of LYVE).

A “biologically active variant or fragment polypeptide” refers to a polypeptide exhibiting at least one, preferably all, of the biological activities of the reference polypeptide such as sLYVE-1, provided the biologically active fragment retains the capacity of reducing the FGF2-induced lymphoangiogenesis. The biologically active fragment may for example be characterized in that it is capable of inhibiting LEC tubulogenesis (see Example); and/or inhibiting LEC proliferation (see Example); and/or inhibiting LEC migration (see Example).

An “LYVE-1 antagonist” refers to a molecule (natural or synthetic) capable of neutralizing, blocking, inhibiting, abrogating, reducing or interfering with the activities of LYVE-1 including, for example, reduction or blocking of LYVE-1 receptor activation, reduction or blocking of LYVE-1 downstream molecular signaling. LYVE-1 antagonists include antibodies and antigen-binding fragments thereof, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. Antagonists also include small molecule inhibitors of a protein and receptor molecules and derivatives which bind specifically to FGF2 thereby sequestering its binding to its LYVE-1 receptor such as soluble LYVE-1 receptors or fusions proteins (including immunoadhesins, e.g., LYVE-1Fc molecules), antagonist variants of the protein, siRNA molecules directed to a protein, antisense molecules directed to a protein, aptamers, and ribozymes against a protein. For instance, the LYVE-1 antagonist may be a molecule which binds to LYVE-1 and neutralizes, blocks, inhibits, abrogates, reduces or interferes with a biological activity of FGF2 (such as stimulation of lymphangiogenesis by promoting the Lymphatic Endothelial Cells (LEC) tubulogenesis and/or promoting the LEC proliferation and/or promoting the LEC migration). Alternatively, the LYVE-1 antagonist may be a molecule which binds to FGF2 and neutralizes, blocks, inhibits, abrogates, reduces or interferes with a biological activity of FGF2.

As used herein, the term “lymphangiogenesis” refers to growth of new lymphatic vessels. Accordingly, a “lymphangiogenic factor” is a growth factor and “lymphangiogenic therapy” is therapy which stimulates the development of lymphatic vessels. Lymphangiogenic factors disclosed herein include, but are not limited to, VEGF-C, VEGF-D and FGF2. It follows that “anti-lymphangiogenic” relates to inhibition of lymphangiogenesis.

By “metastasis” or “tumor metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream or lymphatics, and stopping at a distant site. At the new site, the cells establish a blood supply and can grow to form a life-threatening mass. In certain embodiments, the term metastatic tumor refers to a tumor that is capable of metastasizing, but has not yet metastasized to tissues or organs elsewhere in the body. In certain embodiments, the term metastatic tumor refers to a tumor that has metastasized to tissues or organs elsewhere in the body.

By “primary tumor” or “primary cancer” is meant the original cancer and not a metastatic lesion located in another tissue, organ, or location in the subject's body.

As used herein, the term “subject” denotes a mammal, such as a rodent, a feline, a canine, and a primate. Preferably, a subject according to the invention is a human.

### Therapeutic Methods and Uses

The present invention provides methods and compositions (such as pharmaceutical compositions) for preventing or treating a pathological condition associated with lymphangiogenesis. The present invention also provides methods and compositions for inhibiting or preventing tumor metastasis or tumoral lymphangiogenesis.

According to a first aspect, the present invention relates to a Lymphatic Vessel Endothelial Hyaluronan Receptor-1 (LYVE-1) antagonist for use in the prevention or the treatment of a pathological condition associated with lymphangiogenesis.

In a second aspect, the present invention also relates to a LYVE-1 antagonist for use in the prevention of tumor metastasis.

In one embodiment, the LYVE-1 antagonist is an inhibitor of the interaction between LYVE-1 and FGF2.

The terms “blocking the interaction”, “inhibiting the interaction” or “inhibitor of the interaction” are used herein to mean preventing or reducing the direct or indirect association of one or more molecules, peptides, proteins, enzymes or receptors; or preventing or reducing the normal activity of one or more molecules, peptides, proteins, enzymes, or receptors.

Thus, the term “inhibitor of the interaction between LYVE-1 and FGF2” refers to a molecule which can prevent the interaction between LYVE-1 and FGF2 by competition or by fixing to one of the molecules.

Accordingly, the LYVE-1 antagonist may be a molecule which binds to LYVE-1 or FGF2 selected from the group consisting of antibodies, aptamers, polypeptides and small organic molecules.

In one embodiment, the LYVE-1 antagonist is an isolated LYVE-1 receptor polypeptide.

As used herein, the term “LYVE-1 receptor polypeptide” refers to a polypeptide that specifically bind to FGF2 can be used as LYVE-1 antagonists that bind to and sequester the FGF2 protein, thereby preventing it from signaling.

In a particular embodiment, the LYVE-1 receptor polypeptide is soluble. A soluble LYVE-1 receptor polypeptide exerts an inhibitory effect on the biological activity of the FGF2 protein by binding to the protein, thereby preventing it from binding to LYVE-1 present on the surface of target cells. It is undesirable for a LYVE-1 receptor polypeptide not to become associated with the cell membrane. In a preferred embodiment, the soluble LYVE-1 receptor polypeptide lacks any amino acid sequences corresponding to the transmembrane and intracellular domains from the LYVE-1 receptor from which it is derived.

In a preferred embodiment, said polypeptide is a soluble LYVE-1 (sLYVE-1) or a functional equivalent thereof.

The terms “soluble LYVE-1” or “sLYVE-1”, as used herein, refer to a polypeptide comprising or consisting of the extracellular region of the LYVE-1 receptor or a fragment thereof. For example, sLYVE-1 may include all the extracellular domain of human LYVE-1 (i.e. a polypeptide comprising or consisting of the amino acid sequence ranging from positions 1-232 of human LYVE-1 as shown by SEQ ID NO: 1 above).

A “functional equivalent of sLYVE-1” is a molecule which is capable of binding to FGF2, preferably which is capable of specifically binding to FGF2. The term “functional equivalent” includes fragments and variants of sLYVE-1 as above described. As used herein, “binding specifically” means that the biologically active fragment has high affinity for FGF2 but not for control proteins. Specific binding may be measured by a number of techniques such as ELISA, flow cytometry, western blotting, or immunoprecipitation. Preferably, the functionally equivalent specifically binds to FGF2 at nanomolar or picomolar levels.

By “biological activity” of a functional equivalent of the extracellular region of the LYVE-1 is meant (i) the capacity to bind to FGF2; and/or (ii) the capacity to reduce FGF2-induced lymphoangiogenesis; and/or (iii) the capacity to reduce the (Lymphatic Endothelial Cells) LEC tubulogenesis; and/or (iv) the capacity to reduce the LEC proliferation and/or (v) the capacity to reduce the LEC migration, (vi) the capacity to inhibit the effect of FGF2 on lymphangiogenesis in vivo.

The skilled in the art can easily determine whether a functional equivalent of the extracellular region of the LYVE-1 is biologically active. To check whether the newly generated polypeptides bind to FGF2 and/or reduce FGF2-induced lymphoangiogenesis in the same way than the initially characterized polypeptide sLYVE-1 (a polypeptide comprising or consisting of the amino acid sequence ranging from positions 1-232 of SEQ ID NO: 1) a binding assay, a cell proliferation assay or a cell migration assay (see in Example) may be performed with each polypeptide. Additionally, a time-course and a dose-response performed in vitro or in vivo (e.g. by using a corneal micropocket assay as described in the Examples section) will determine the optimal conditions for each polypeptide.

Thus, the polypeptide according to the invention encompasses polypeptides comprising or consisting of fragments of the extracellular region of the LYVE-1, provided the fragments are biologically active. In the frame of the invention, the biologically active fragment may for example comprise at least 15, 20, 25, 50, 75, 100, 150 or 200 consecutive amino acids of the extracellular region of the LYVE-1.

In one embodiment, the polypeptides of the invention may comprise a tag. A tag is an epitope-containing sequence which can be useful for the purification of the polypeptides. It is attached to by a variety of techniques such as affinity chromatography, for the localization of said peptide or polypeptide within a cell or a tissue sample using immunolabeling techniques, the detection of said polypeptide by immunoblotting etc. Examples of tags commonly employed in the art are the GST (glutathion-S-transferase)-tag, the FLAG™-tag, the Strep-tag™, V5 tag, myc tag, His tag (which typically consists of six histidine residues), etc.

In another embodiment, the polypeptides of the invention may comprise chemical modifications improving their stability and/or their biodisponibility. Such chemical modifications aim at obtaining polypeptides with increased protection of the polypeptides against enzymatic degradation in vivo, and/or increased capacity to cross membrane barriers, thus increasing its half-life and maintaining or improving its biological activity. Any chemical modification known in the art can be employed according to the present invention. Such chemical modifications include but are not limited to:
replacement(s) of an amino acid with a modified and/or unusual amino acid, e.g. a replacement of an amino acid with an unusual amino acid like Nle, Nva or Orn; and/or
modifications to the N-terminal and/or C-terminal ends of the peptides such as e.g. N-terminal acylation (preferably acetylation) or desamination, or modification of the C-terminal carboxyl group into an amide or an alcohol group;
modifications at the amide bond between two amino acids: acylation (preferably acetylation) or alkylation (preferably methylation) at the nitrogen atom or the alpha carbon of the amide bond linking two amino acids;
modifications at the alpha carbon of the amide bond linking two amino acids such as e.g. acylation (preferably acetylation) or alkylation (preferably methylation) at the alpha carbon of the amide bond linking two amino acids.
chirality changes such as e.g. replacement of one or more naturally occurring amino acids (L enantiomer) with the corresponding D-enantiomers;
retro-inversions in which one or more naturally-occurring amino acids (L-enantiomer) are replaced with the corresponding D-enantiomers, together with an inversion of the amino acid chain (from the C-terminal end to the N-terminal end);
azapeptides, in which one or more alpha carbons are replaced with nitrogen atoms; and/or
betapeptides, in which the amino group of one or more amino acid is bonded to the β carbon rather than the α carbon.

In another embodiment, adding dipeptides can improve the penetration of a circulating agent in the eye through the blood retinal barrier by using endogenous transporters.

Another strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 60 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

Alternatively, a nucleic acid encoding a polypeptide of the invention (such as sLYVE-1) or a vector comprising such nucleic acid or a host cell comprising such expression vector may be used in the prevention or treatment of a pathological condition associated with lymphangiogenesis or used in the prevention of tumor metastasis.

Nucleic acids of the invention may be produced by any technique known per se in the art, such as, without limitation, any chemical, biological, genetic or enzymatic technique, either alone or in combination(s).

Expression vectors of the invention are well known in the art (since they are easily constructed using conventional methods or are commercially available) and are disclosed below (see the section “Inhibitors of LYVE-1 gene expression”).

In another particular embodiment, the polypeptide is a LYVE-1 fusion protein.

As used herein, “LYVE-1 fusion protein” means a protein comprising a soluble LYVE-1 polypeptide fused to a heterologous polypeptide (i.e. polypeptide derived from an unrelated protein, for example, from an immunoglobulin protein).

As used herein, the terms “fused” and “fusion” are used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments may be physically or spatially separated by, for example, in-frame linker sequence.

As used herein, the term “fusion protein” means a protein comprising a first polypeptide linearly connected, via peptide bonds, to a second, polypeptide.

As used herein, the term “LYVE-1 fusion protein” refers to a polypeptide comprising the extracellular region of the LYVE-1 receptor or a fragment thereof fused to heterologous polypeptide. The LYVE-1 fusion protein will generally share at least one biological property in common with sLYVE-1 (as described above).

An example of a LYVE-1 fusion protein is a LYVE-1 immunoadhesin.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

The term “LYVE-1 immunoadhesin” is used interchangeably with the term “LYVE-1-immunoglobulin chimera”, and refers to a chimeric molecule that combines at least a fragment of an LYVE-1 molecule (native or variant) with an immunoglobulin sequence. For instance, the LYVE-1 immunoadhesin comprises the extracellular domain (ECD) of LYVE-1 or a fragment thereof sufficient to bind to LYVE-1 ligand (FGF2).

In a preferred embodiment, the LYVE-1 immunoadhesin comprises a polypeptide comprising or consisting of the amino acid sequence ranging from positions 1-232 of SEQ ID NO: 1 and an immunoglobulin sequence.

The immunoglobulin sequence preferably, but not necessarily, is an immunoglobulin constant domain (Fc region). Immunoadhesins can possess many of the valuable chemical and biological properties of human antibodies. Since immunoadhesins can be constructed from a human protein sequence with a desired specificity linked to an appropriate human immunoglobulin hinge and constant domain (Fc) sequence, the binding specificity of interest can be achieved using entirely human components. Such immunoadhesins are minimally immunogenic to the patient, and are safe for chronic or repeated use. In one embodiment, the Fc region is a native sequence Fc region. In another embodiment, the Fc region is a variant Fc region. In still another embodiment, the Fc region is a functional Fc region. The LYVE-1 portion and the immunoglobulin sequence portion of the LYVE-1 immunoadhesin may be linked by a minimal linker. The immunoglobulin sequence preferably, but not necessarily, is an immunoglobulin constant domain. The immunoglobulin moiety in the chimeras of the present invention may be obtained from IgG1, IgG2, IgG3 or IgG4 subtypes, IgA, IgE, IgD or IgM, but preferably IgG1 or IgG3.

As used herein, the term “Fc region” is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof.

A “functional Fc region” possesses an “effector function” of a native sequence Fc region. Exemplary “effector functions” include Clq binding; complement dependent cytotoxicity; Fc receptor binding; antibody-dependent cell-mediated cytotoxicity (ADCC); phagocytosis; down regulation of cell surface receptors (e.g. B cell receptor; BCR), etc.

A “native sequence Fc region” comprises an amino acid sequence identical to the amino acid sequence of an Fc region found in nature. Native sequence human Fc regions include a native sequence human IgGi Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

The polypeptides of the invention may be produced by any suitable means, as will be apparent to those of skill in the art. In order to produce sufficient amounts of a sLYVE-1 or functional equivalents thereof, or a LYVE-1 fusion protein such as a LYVE-1 immunoadhesin for use in accordance with the present invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known.

When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E coli.

In one particular embodiment, the polypeptide such as sLYVE-1 or the LYVE-1 fusion protein such as a LYVE-1 immunoadhesin is a glycosylated polypeptide.

Indeed, the majority of protein-based biopharmaceuticals bare some form of post-translational modification which can profoundly affect protein properties relevant to their therapeutic application. Protein glycosylation represents the most common modification (about 50% of human proteins are glycosylated). Glycosylation can introduce considerable heterogeneity into a protein composition through the generation of different glycan structures on the proteins within the composition. Such glycan structures are made by the action of diverse enzymes of the glycosylation machinery as the glycoprotein transits the Endoplasmatic Reticulum (ER) and the Golgi-Complex (glycosylation cascade). The nature of the glycan structure(s) of a protein has impact on the protein's folding, stability, life time, trafficking, pharmaco-dynamics, pharmacokinetics and immunogenicity. The glycan structure has great impact on the protein's primary functional activity. Glycosylation can affect local protein structure and may help to direct the folding of the polypeptide chain. One important kind of glycan structures are the so called N-glycans. They are generated by covalent linkage of an oligosaccharide to the amino (N)-group of asparagin residues in the consensus sequence NXS/T of the nascent polypeptide chain. N-glycans may further participate in the sorting or directing of a protein to its final target: the N-glycan of an antibody, for example, may interact with complement components. N-glycans also serve to stabilize a glycoprotein, for example, by enhancing its solubility, shielding hydrophobic patches on its surface, protecting from proteolysis, and directing intra-chain stabilizing interactions. Glycosylation may regulate protein half-life, for example, in humans the presence of terminal sialic acids in N-glycans may increase the half-life of proteins, circulating in the blood stream.

As used herein, the term “glycoprotein” refers to any protein having one or more N-glycans attached thereto. Thus, the term refers both to proteins that are generally recognized in the art as a glycoprotein and to proteins which have been genetically engineered to contain one or more N-linked glycosylation sites. As used herein, the terms “N-glycan” and “glycoform” are used interchangeably and refer to an N-linked oligosaccharide, for example, one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. The predominant sugars found on glycoproteins are glucose, galactose, mannose, fucose, N-acetylgalactosamine (GalNAc), N-acetylglucosamine (GlcNAc) and sialic acid (e.g., N-acetyl-neuraminic acid (NANA)). The processing of the sugar groups occurs co-translationally in the lumen of the ER and continues post-translationally in the Golgi apparatus for N˜linked glycoproteins.

A number of yeasts, for example, Pichia pastoris, Yarrowia lipolytica and Saccharomyces cerevisiae are recently under development to use the advantages of such systems but to eliminate the disadvantages in respect to glycosylation. Several strains are under genetical development to produce defined, human-like glycan structures on a protein. Methods for genetically engineering yeast to produce human-like N-glycans are described in U.S. Pat. Nos. 7,029,872 and 7,449,308 along with methods described in U.S. Published Application Nos. 20040230042, 20050208617, 20040171826, 20050208617, and 20060286637. These methods have been used to construct recombinant yeast that can produce therapeutic glycoproteins that have predominantly human-like complex or hybrid N-glycans thereon instead of yeast type N-glycans. As previously described, human-like glycosylation is primarily characterized by “complex” N-glycan structures containing N-acetylglusosamine, galactose, fucose and/or N-acetylneuraminic acid. Thus, several strains of yeasts have been genetically engineered to produce glycoproteins comprising one or more human-like complex or human-like hybrid N-glycans such as GlcNAcMan3GlcNAc2.

In another embodiment, the LYVE-1 antagonist is an antibody (the term including antibody fragment or portion) that can block the interaction of LYVE-1 with FGF2.

In preferred embodiment, the LYVE-1 antagonist may consist in an antibody directed against the LYVE-1 or FGF2, in such a way that said antibody impairs the binding of a FGF2 to LYVE-1 (“neutralizing antibody”).

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of LYVE-1. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the recombinant LYVE-1 may be provided by expression with recombinant cell lines. Recombinant form of LYVE-1 may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., /. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

Then, for this invention, once antibodies which bind to LYVE-1 (preferably which specifically bind) have been obtained, neutralizing antibodies of LYVE-1 are selected. Accordingly, in a particular embodiment, the antibody which binds to LYVE-1 is a neutralizing anti-LYVE-1 antibody (i.e. an antibody which blocks the activity of LYVE-1 leading to the inhibition of FGF2-induced lymphoangiogenesis.

In another embodiment, the LYVE-1 antagonist is an aptamer directed against LYVE-1 or FGF2. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Then, for this invention, neutralizing aptamers of LYVE-1 are selected as above described (for their capacity to inhibit FGF2-induced lymphoangiogenesis).

In another embodiment, the LYVE-1 antagonist is a small organic molecule. As used herein, the term “small organic molecule” refers to a molecule of size comparable to those organic molecules generally sued in pharmaceuticals. The term excludes biological macromolecules (e.g.; proteins, nucleic acids, etc.); preferred small organic molecules range in size up to 2000 Da, and most preferably up to about 1000 Da.

In still another embodiment, the LYVE-1 antagonist is an inhibitor of LYVE-1 gene expression. An “inhibitor of expression” refers to a natural or synthetic compound that has a biological effect to inhibit the expression of a gene. Therefore, an “inhibitor of LYVE-1 gene expression” denotes a natural or synthetic compound that has a biological effect to inhibit the expression of LYVE-1 gene.

In a preferred embodiment of the invention, said inhibitor of LYVE-1 gene expression is a siRNA, an antisense oligonucleotide or a ribozyme.

Inhibitors of LYVE-1 gene expression for use in the present invention may be based on antisense oligonucleotide constructs. Anti-sense oligonucleotides, including anti-sense RNA molecules and anti-sense DNA molecules, would act to directly block the translation of LYVE-1 mRNA by binding thereto and thus preventing protein translation or increasing mRNA degradation, thus decreasing the level of LYVE-1, and thus activity, in a cell. For example, antisense oligonucleotides of at least about 15 bases and complementary to unique regions of the mRNA transcript sequence encoding LYVE-1 can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of LYVE-1 gene expression for use in the present invention. LYVE-1 gene expression can be reduced by using small double stranded RNA (dsRNA), or a vector or construct causing the production of a small double stranded RNA, such that LYVE-1 gene expression is specifically inhibited (i.e. RNA interference or RNAi). Methods for selecting an appropriate dsRNA or dsRNA-encoding vector are well known in the art for genes whose sequence is known (e.g. see Tuschi, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Examples of said siRNAs against human LYVE-1 include, but are not limited to, those purchased by Dharmacon ON-TARGET plus SMART pool, ON-TARGETplus siRNA-Human XLKD1 no. J-020129.

Ribozymes can also function as inhibitors of LYVE-1 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of LYVE-1 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Antisense oligonucleotides, siRNAs and ribozymes useful as inhibitors of LYVE-1 gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating the transfer of the antisense oligonucleotide, siRNA or ribozyme nucleic acid to the cells and preferably cells expressing LYVE-1. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Chiffon, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

Examples of pathological conditions or disorders associated with lymphangiogenesis (i.e. abnormal lymphangiogenesis) include, without limitation, cancer, eye diseases (such as corneal graft rejection, age-related macular degeneration and diabetic retinopathy) and inflammatory diseases (such as rheumatoid arthritis and psoriasis).

The terms “cancer” and “cancerous” refer to or describe the physiological condition in mammals that is typically characterized by unregulated cell growth/proliferation.

Examples of neoplastic disorders to be treated with an LYVE-1 antagonist include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, gastric cancer, melanoma, and various types of head and neck cancer.

Non-cancerous disorders to be treated with an LYVE-1 antagonist include, but are not limited to, eye diseases and inflammatory diseases. More particularly, examples of eye diseases to be treated with an LYVE-1 antagonist include, but are not limited to, diabetic and other proliferative retinopathies including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease.

Other non-cancerous disorders that are amenable to treatment with LYVE-1 antagonists useful in the invention, include but are not limited to, e.g., lymphoedema, undesired or aberrant hypertrophy, arthritis, rheumatoid arthritis (RA), psoriasis, psoriatic plaques, sarcoidosis, atherosclerosis, atherosclerotic plaques, edema from myocardial infarction, vascular restenosis, arteriovenous malformations (AVM), meningioma, hemangioma, angiofibroma, thyroid hyperplasias (including Grave's disease), corneal and other tissue transplantation, chronic inflammation, lung inflammation, acute lung injury/ARDS, sepsis, primary pulmonary hypertension, malignant pulmonary effusions, cerebral edema (e.g., associated with acute stroke/closed head injury/trauma), synovial inflammation, pannus formation in RA, myositis ossificans, hypertropic bone formation, osteoarthritis (OA), refractory ascites, polycystic ovarian disease, endometriosis, 3rd spacing of fluid diseases (pancreatitis, compartment syndrome, burns, bowel disease), uterine fibroids, premature labor, chronic inflammation such as IBD (Crohn's disease and ulcerative colitis), renal allograft rejection, inflammatory bowel disease, nephrotic syndrome, undesired or aberrant tissue mass growth (non-cancer), obesity, adipose tissue mass growth, hemophilic joints, hypertrophic scars, inhibition of hair growth, Osier-Weber syndrome, pyogenic granuloma retrolental fibroplasias, scleroderma, trachoma, vascular adhesions, synovitis, dermatitis, preeclampsia, ascites, pericardial effusion (such as that associated with pericarditis), and pleural effusion.

In another aspect, the present invention provides a method of inhibiting lymphangiogenesis using a therapeutically effective amount of an LYVE-1 antagonist.

In another aspect, the present invention provides a method of inhibiting lymphangiogenesis comprising administering a therapeutically effective amount of an LYVE-1 antagonist to a subject in need thereof.

Examples of pathological conditions or disorders associated with lymphangiogenesis (i.e. abnormal lymphangiogenesis) include, without limitation, cancer, eye diseases (such as corneal graft rejection, age-related macular degeneration and diabetic retinopathy) and inflammatory diseases (such as rheumatoid arthritis and psoriasis).

In still another aspect, the present invention provides a method of inhibiting or preventing tumoral lymphangiogenesis in a subject comprising administering to the subject a therapeutically effective amount of an LYVE-1 antagonist.

In a further aspect, the present invention also provides a method of inhibiting or preventing tumor metastasis in a subject comprising administering to the subject a therapeutically effective amount of LYVE-1 antagonist.

In one embodiment, the subject may have developed or be at risk for developing tumor metastasis. Such metastasis may be in the lymphatic system or in a distant organ.

The present invention further contemplates a method of preventing or treating cancer in a subject comprising administering to the subject a therapeutically effective amount of an LYVE-1 antagonist.

In one aspect, the present invention provides a method of inhibiting tumor growth in a subject comprising administering a therapeutically effective amount of an LYVE-1 antagonist.

By a “therapeutically effective amount” of a LYVE-1 antagonist as above described is meant a sufficient amount of the antagonist to prevent or treat a pathological cobndition associated with lymphangiogenesis (e.g. cancer or eye diseases). It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The terms “treat”, “treating” or “treatment” refer to both therapeutic treatment and prophylactic or preventative measures, wherein the aim is to prevent or ameliorate cancer or slow down (lessen) cancer progression. Those in need of treatment include those already with the disorder as well as those in which the disorder is to be prevented.

The terms “preventing”, “prevention”, “preventative” or “prophylactic” refer to keeping from occurring, or to hinder, defend from, or protect from the occurrence of a condition, disease, disorder, or phenotype, including an abnormality or symptom. A subject in need of prevention may be prone to develop the condition.

### Pharmaceutical Compositions of the Invention

The LYVE-1 antagonist as described above may be combined with pharmaceutically acceptable excipients, and optionally sustained-release matrices, such as biodegradable polymers, to form therapeutic compositions.

Accordingly, the present invention relates to a pharmaceutical composition comprising a LYVE-1 antagonist according to the invention and a pharmaceutically acceptable carrier.

The present invention also relates to a pharmaceutical composition for use in the prevention or treatment of a pathological condition associated with lymphangiogenesis comprising a LYVE-1 antagonist according to the invention and a pharmaceutically acceptable carrier.

The present invention further relates to a pharmaceutical composition for preventing tumor metastasis comprising a LYVE-1 antagonist according to the invention and a pharmaceutically acceptable carrier.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In therapeutic applications, compositions are administered to a patient already suffering from a disease, as described, in an amount sufficient to cure or at least partially stop the symptoms of the disease and its complications. An appropriate dosage of the pharmaceutical composition is readily determined according to any one of several well-established protocols. For example, animal studies (for example on mice or rats) are commonly used to determine the maximal tolerable dose of the bioactive agent per kilogram of weight. In general, at least one of the animal species tested is mammalian. The results from the animal studies can be extrapolated to determine doses for use in other species, such as humans for example. What constitutes an effective dose also depends on the nature and severity of the disease or condition, and on the general state of the patient's health.

In prophylactic applications, compositions containing, for example LYVE-1 receptor antagonists, are administered to a patient susceptible to or otherwise at risk of a pathological condition associated with lymphoangiogenesis (e.g. cancer or eye disease). Such an amount is defined to be a “prophylactically effective” amount or dose. In this use, the precise amount depends on the patient's state of health and weight.

In both therapeutic and prophylactic treatments, the antagonist contained in the pharmaceutical composition can be administered in several dosages or as a single dose until a desired response has been achieved. The treatment is typically monitored and repeated dosages can be administered as necessary. Compounds of the invention may be administered according to dosage regimens established whenever inactivation of LYVE-1 is required.

The daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 10 mg/kg of body weight per day. It will be understood, however, that the specific dose level and frequency of dosage for any particular patient may be varied and will depend upon a variety of factors including the activity of the specific compound employed, the metabolic stability, and length of action of that compound, the age, the body weight, general health, sex, diet, mode and time of administration, rate of excretion, drug combination, the severity of the particular condition, and the host undergoing therapy.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

The appropriate unit forms of administration include forms for oral administration, such as tablets, gelatine capsules, powders, granules and solutions or suspensions to be taken orally, forms for sublingual and buccal administration, aerosols, implants, forms for subcutaneous, intramuscular, intravenous, intranasal or intraocular administration and forms for rectal administration.

In the pharmaceutical compositions of the present invention, the active principle is generally formulated as dosage units containing from 0.5 to 1000 mg, preferably from 1 to 500 mg, more preferably from 2 to 200 mg of said active principle per dosage unit for daily administrations.

When preparing a solid composition in the form of tablets, a wetting agent such as sodium laurylsulfate can be added to the active principle optionally micronized, which is then mixed with a pharmaceutical vehicle such as silica, gelatine, starch, lactose, magnesium stearate, talc, gum arabic or the like. The tablets can be coated with sucrose, with various polymers or other appropriate substances or else they can be treated so as to have a prolonged or delayed activity and so as to release a predetermined amount of active principle continuously.

A preparation in the form of gelatin capsules is obtained by mixing the active principle with a diluent such as a glycol or a glycerol ester and pouring the mixture obtained into soft or hard gelatine capsules.

A preparation in the form of a syrup or elixir can contain the active principle together with a sweetener, which is preferably calorie-free, methyl-paraben and propylparaben as an antiseptic, a flavoring and an appropriate color.

The water-dispersible powders or granules can contain the active principle mixed with dispersants or wetting agents, or suspending agents such as polyvinyl-pyrrolidone, and also with sweeteners or taste correctors.

Rectal administration is effected using suppositories prepared with binders which melt at the rectal temperature, for example cacao butter or polyethylene glycols.

Parenteral, intranasal or intraocular administration is effected using aqueous suspensions, isotonic saline solutions or sterile and injectable solutions which contain pharmacologically compatible dispersants and/or wetting agents, for example propylene glycol, butylene glycol, or polyethylene glycol.

Thus a cosolvent, for example an alcohol such as ethanol or a glycol such as polyethylene glycol or propylene glycol, and a hydrophilic surfactant such as Tween® 80, can be used to prepare an aqueous solution injectable by intravenous route. The active principle can be solubilized by a triglyceride or a glycerol ester to prepare an oily solution injectable by intramuscular route.

Transdermal administration is effected using multilaminated patches or reservoirs into which the active principle is in the form of an alcoholic solution.

Administration by inhalation is effected using an aerosol containing for example sorbitan trioleate or oleic acid together with trichlorofluoromethane, dichlorotetrafluoroethane or any other biologically compatible propellant gas.

The active principle can also be formulated as microcapsules or microspheres, optionally with one or more carriers or additives.

Among the prolonged-release forms which are useful in the case of chronic treatments, implants can be used. These can be prepared in the form of an oily suspension or in the form of a suspension of microspheres in an isotonic medium.

The active principle can also be presented in the form of a complex with a cyclodextrin, for example .alpha.-, .beta.- or .gamma.-cyclodextrin, 2-hydroxypropyl-.beta.-cyclodextrin or methyl-.beta.-cyclodextrin.

### Combination Therapies of the Invention

In other embodiments, the LYVE-1 antagonist may be administered to a subject with an appropriate additional therapeutic agent useful in prevention or treatment of the condition from which the patient suffers or is susceptible to; examples of such agents include a chemotherapeutic agent, an immunomodulatory agent, a hormonal agent, an anti-inflammation drug, a steroid, an immune system suppressor, a corticosteroid, etc.

The administration of the LYVE-1 antagonist and the other therapeutic agent, (e.g., a chemotherapeutic agent) can be carried out simultaneously, e.g., as a single composition or as two or more distinct compositions using the same or different administration routes. Alternatively, or additionally, the administration can be done sequentially, in any order. Alternatively, or additionally, the steps can be performed as a combination of both sequentially and simultaneously, in any order. In certain embodiments, intervals ranging from minutes to days, to weeks to months, can be present between the administrations of the two or more compositions. For example, the additional therapeutic agent may be administered first, followed by the LYVE-1 antagonist. However, simultaneous administration or administration of the LYVE-1 antagonist first is also contemplated.

Accordingly, in one aspect, the present invention relates to a pharmaceutical composition comprising a LYVE-1 antagonist according to the invention and an additional therapeutic agent.

In another aspect, the present invention relates to a kit-of-part composition comprising a LYVE-1 antagonist according to the invention and an additional therapeutic agent.

In still another aspect, the present invention relates to a pharmaceutical composition for use in the prevention or treatment of a pathological condition associated with lymphangiogenesis comprising a LYVE-1 antagonist according to the invention and an additional therapeutic agent.

Also provided, is a pharmaceutical composition for use in the prevention of tumor metastasis comprising a LYVE-1 antagonist according to the invention and an additional therapeutic agent.

### Combination LYVE-1 Antagonist with a Chemotherapeutic Compound

It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

Non-limiting examples of chemotherapeutic compounds which can be used in combination treatments of the present invention include, for example, conventional chemotherapeutic, radiotherapeutic and anti-angiogenic agents.

Examplary anti-cancer anti-angiogenic agents inhibit signaling by a receptor tyrosine kinase including but not limited to FGFR (fibroblast growth factor receptor, FGF-1,2), PDGFR (platelet derived growth factor receptor), angiopoïetins receptors (Ang-1,2), HGFR (hepatocytary growth factor receptor), ephrines receptor (Eph), VEGFR1, VEGFR-2,3 PDGFR-α, PDGFR-β, CSF-1R, MET, Flt-3, c-Kit, bcr/abl, p38 alpha and FGFR-1. Further anti-angiogenic agents may include agents that inhibit one or more of the various regulators of VEGF expression and production, such as EGFR, flt-1, KDR HER-2, COX-2, or HIF-1α. Another preferred class of agents includes IMiD (immunomodulatory drugs), analogs derived from thalidomide that have a wide range of effects, including both immune and non-immune related effects. Representatives of the IMiD class include CC-5013 (lenalidomide, Revlimid™), CC-4047 (Actimid™), and ENMD-0995. Another class of anti-angiogenic agent includes cilengitide (EMD 121974, integrin inhibitor), metalloproteinases (MPP) such as marinastat (BB-251). Another class of anti-angiogenic agents includes farnesylation inhibitors such as lonafarnib (Sarasar™), tipifarnib (Zarnestra™). Other anti-angiogenic agents can also be suitable such as Bevacuzimab (mAb, inhibiting VEGF-A, Genentech); IMC-1121B (mAb, inhibiting VEGFR-2, ImClone Systems); CDP-791 (Pegylated DiFab, VEGFR-2, Celltech); 2C3 (mAb, VEGF-A, Peregrine Pharmaceuticals); VEGF-trap (Soluble hybrid receptor VEGF-A, P1GF (placenta growth factor) Aventis/Regeneron). Another preferred class of agents includes the tyrosine kinase inhibitor (TKI) class, including, e.g., PTK-787 (TKI, VEGFR-1,-2, Vatalanib, Novartis); AEE788 (TKI, VEGFR-2 and EGFR, Novartis); ZD6474 (TKI, VEGFR-1,-2,-3, EGFR, Zactima, AstraZeneca); AZD2171 (TKI, VEGFR-1,-2, AstraZeneca); SU11248 (TKI, VEGFR-1, -2, PDGFR, Sunitinib, Pfizer); AG13925 (TKI, VEGFR-1,-2, Pfizer); AG013736 (TKI, VEGFR-1,-2, Pfizer); CEP-7055 (TKI, VEGFR-1,-2,-3, Cephalon); CP-547,632 (TKI, VEGFR-1,-2, Pfizer); GW786024 (TKI, VEGFR-1,-2,-3, GlaxoSmithKline); GW786034 (TKI, VEGFR-1,-2,-3, GlaxoSmithKline); sorafenib (TKI, Bay 43-9006, VEGFR-1,-2, PDGFR Bayer/Onyx); SU4312 (TKI, VEGFR, PDGFR, Pfizer), AMG706 (TKI, VEGFR-1,-2,-3, Amgen), XL647 (TKI, EGFR, HER2, VEGFR, ErbB4, Exelixis), XL999 (TKI, FGFR, VEGFR, PDGFR, Flt-3, Exelixis), PKC412 (TKI, KIT, PDGFR, PKC, FLT3, VEGFR-2, Novartis), AEE788 (TKI, EGFR, HER2, VEGFR, Novartis), OSI-930 (TKI, c-kit, VEGFR, OSI Pharmaceuticals), OSI-817 (TKI, c-kit, VEGFR, OSI Pharmaceuticals), DMPQ (TKI, ERGF, PDGFR, erbB2, p56, pkA, pkC), MLN518 (TKI, FLT3, PDGFR, c-KIT, CT53518, Millennium Pharmaceuticals), lestaurinib (TKI, FLT3, CEP-701, Cephalon), ZD1839 (TKI, EGFR, gefitinib, Iressa, AstraZeneca), OSI-774 (TKI, EGFR, Erlotininb, Tarceva, OSI Pharmaceuticals), lapatinib (TKI, ErbB-2, EGFR, GD-2016, Tykerb, GlaxoSmithKline). Most preferred are tyrosine kinase inhibitors that inhibit one or more receptor tyrosine kinases selected from the group consisting of VEGFR-1, VEGFR-2, VEGFR-3, PDGFR-α, β, Flt-3, c-Kit, p38 alpha, MET, c-RAF, b-RAF, bcr/abl and FGFR-1.

Further anti-cancer agents include alkylating agents, cytotoxic antibiotics such as topoisomerase I inhibitors, topoisomerase II inhibitors, plant derivatives, RNA/DNA antimetabolites, and antimitotic agents. Preferred examples may include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, taxol, gemcitabine, navelbine, transplatinum, 5-fluorouracil, vincristin, vinblastin and methotrexate, or any analog or derivative variant of the foregoing.

### Combination LYVE-1 Antagonist with a VEGFR3 Antagonist

The present invention is based in part on the discovery that administration of LYVE-1 antagonist (e.g., sLYVE-1 or siRNA against LYVE-1) results in an in vivo inhibition of FGF2-induced lymphangiogenesis but not VEGF-C-induced lymphangiogenesis. Accordingly administration of combinations of an LYVE-1 antagonist and a VEGFR3 antagonist are useful for prevention or treating pathological conditions and disorders associated with lymphangiogenesis (e.g. cancer or eye diseases) or for preventing tumor metastasis.

As previously mentioned VEGFR-3 mediates lymphangiogenesis in response to VEGF-C. Thus, it should be reminded that VEGF-C is a member of a structurally related VEGF family of angiogenic regulators. In addition to an angiogenic activity, VEGF-C appears to be involved in regulation of lymphangiogenesis via its binding to VEGFR-3. The angiogenesis induced by VEGF-C in tumors can promote solid tumor growth and metastatic spread, and the lymphangiogenesis induced by VEGF-C can promote metastatic spread of tumor cells to the lymphatic vessels and lymph nodes. Furthermore, clinicopathological data indicates a role for this growth factor in a range of prevalent human cancers.

Recently, it has been proposed that intervention and targeting of the FGF-2- and VEGF-C-induced angiogenic and lymphangiogenic synergism could be potentially important approaches for cancer therapy and prevention of metastasis (Cao et al. 2012)

“Vascular endothelial growth factor receptor-3” “VEGFR3” or “Flt4” is an endothelial specific receptor tyrosine kinase well known in the art, regulated by members of the vascular endothelial growth factor family. VEGF-C and VEGF-D are both ligands for VEGFR3.

A “VEGFR3 antagonist” refers to a molecule capable of neutralizing, blocking, inhibiting, abrogating, reducing or interfering with the activities of VEGFR3 including, for example, reduction or blocking of VEGFR3 receptor activation, reduction or blocking of VEGFR3 downstream molecular signaling, disruption or blocking of VEGFR3 ligand (e.g., VEGF-C or VEGF-D) binding to VEGFR3. VEGFR-3 antagonists encompassed by the present invention can be any antagonists. Non-limiting examples of useful antagonists include, e.g., antagonist antibodies and fragments thereof binding to VEGFR3 or its ligands VEGF-C and VEGF-D, soluble polypeptides that inhibit the activity of VEGFR-3 (e.g., an extracellular domain of a VEGFR-3 protein or a derivative thereof), small molecule inhibitors (e.g., small molecule inhibitors of kinases and/or signaling pathways relevant for VEGFR-3 signal transduction), and inhibitors of VEGFR-3 expression or VEGF-C expression and VEGF-D expression (i.e. (e.g., siRNAs, shRNAs, antisense oligonucleotides, ribozymes, etc.).

In one embodiment, LYVE-1 and VEGFR-3 antagonists can be administered together with a radiation treatment and/or with one or more additional compound(s) useful for inhibiting lymphangiogenesis and/or tumor metastasis (as such previously described).

Anti-VEGFR3 Antibodies

In one embodiment, the VEGFR-3 antagonist useful in the methods of the invention is an anti-VEGFR-3 antibody or an antigen-binding portion thereof.

In a particular embodiment, the anti-VEGFR-3 antibody is IMC-3C5.

Other examples of anti-VEGFR-3 antibodies are described in the international patent applications WO2012/033696.

VEGFR-3 Receptor Polypeptides

In one embodiment, the VEGFR-3 antagonist useful in the methods of the invention is a soluble VEGFR3.

VEGFR-3 receptor polypeptides or fragments thereof, that specifically bind to a VEGF-C and/or VEGF-D can be used as VEGFR-3 antagonists that bind to and sequester the VEGF-C and/or the VEGF-D proteins, thereby preventing it from signaling. Preferably, the VEGFR-3 is soluble. A soluble VEGFR-3 receptor polypeptide exerts an inhibitory effect on the biological activity of the VEGF-C and/or VEGF-D proteins by binding to the protein(s), thereby preventing it from binding to its natural receptors present on the surface of target cells. Preferably, the soluble VEGFR-3 lacks any amino acid sequences corresponding to the transmembrane and intracellular domains from the VEGF-3 receptor from which it is derived.

The VEGFR-3 receptor polypeptides may be a fusion protein in which the amino acid sequences derived from the receptor proteins (e.g. the Ig-like domains derived from VEGFR-3) are linked to amino acids from an unrelated protein, for example, immunoglobulin sequences. In a preferred embodiment, the amino acid sequences derived from the receptor protein(s) are fused to an Fc portion of an immunoglobulin. Other amino acid sequences to which Ig-like domains may be fused will be apparent to the skilled person in the art.

Examples of soluble VEGF receptor molecules which sequester VEGF-C, thereby inhibiting VEGF-C activity or signaling via VEGFR-2 and VEGFR-3, are disclosed in WO2000/023565, WO2000/021560, WO2002/060950 and WO 2005/087808. Such inhibitors of VEGF-C activity include soluble VEGFR-2-, VEGFR-3- and NRP-2 derived traps. In a preferred embodiment, the VEGF-C antagonist is a soluble VEGF-C receptor molecule. A preferred VEGF-C receptor molecule is a polypeptide comprising a portion of the extracellular domain of VEGFR-3, the portion comprising at least Ig-like domains 1-3 of the extracellular domain and lacking Ig-like domains 4-7 and the polypeptide lacking any transmembrane domain. These constructs are described in more detail in WO 2002/060950.

One particular example of a soluble VEGFR-3 is VGX-300.

TKI that Inhibits VEGFR-3 Activity

In one embodiment, the VEGFR-3 antagonist useful in the methods of the invention is a tyrosine kinase inhibitor (TKI) that inhibits VEGFR-3 activity.

A “TKI that inhibits VEGFR-3 activity” means an inhibitor of receptor tyrosine kinase activity that selectively or non-selectively reduces the tyrosine kinase activity of a VEGFR-3 receptor. Such an inhibitor generally reduces VEGFR-3 tyrosine kinase activity without significantly effecting the expression of VEGFR-3 and without effecting other VEGFR-3 activities such as ligand-binding capacity. A VEGFR-3 kinase inhibitor can be a molecule that directly binds the VEGFR-3 catalytic domain, for example, an ATP analog. A VEGFR-3 kinase inhibitor can bind the VEGFR-3 catalytic domain through one or more hydrogen bonds similar to those anchoring the adenine moiety of ATP to VEGFR-3 (Engh et al., J. Biol. Chem. 271:26157-26164 (1996); Tong et al., Nature Struc. Biol. 4:311-316 (1997); and Wilson et al., Chem. Biol. 4:423-431 (1997)). A VEGFR-3 kinase inhibitor also can bind the hydrophobic pocket adjacent to the adenine binding site (Mohamedi et al., EMBO J. 17:5896-5904 (1998); Tong et al., supra, 1997; and Wilson et al., supra, 1997).

VEGFR-3 kinase inhibitors useful in the invention include specific VEGFR-3 kinase inhibitors such as indolinones that differentially block VEGF-C and VEGF-D induced VEGFR-3 kinase activity compared to that of VEGFR-2. Such specific VEGFR-3 kinase inhibitors, for example, MAE106 and MAZ51 can be prepared as described in Kirkin et al., Eur. J. Biochem. 268:5530-5540 (2001). Additional VEGFR-3 kinase inhibitors, including specific, selective and non-selective inhibitors, are known in the art or can be identified using one of a number of well known methods for assaying for receptor tyrosine kinase inhibition.

Anti-VEGF-C Antibodies

In one embodiment, the VEGFR-3 antagonist useful in the methods of the invention is an anti-VEGF-C antibody or an antigen-binding portion thereof.

The term “anti-VEGF-C antibody” refers to an antibody that binds to VEGF-C or a biologically active fragment thereof, e.g. the mature fully-processed form, with sufficient affinity and specificity. In a certain embodiment, an anti-VEGF-C antibody is capable of binding VEGF-C with sufficient affinity such that the antibody is useful as a diagnostic and/or therapeutic agent targeting VEGF-C.

One example of an anti-VEGF-C antibody is a monoclonal antibody is VGX-100.

Other examples of anti-VEGF-C antibodies are described in the international patent applications WO2011/125719 and WO 2011/071577.

Anti-VEGF-D Antibodies

In one embodiment, the VEGFR-3 antagonist useful in the methods of the invention is an anti-VEGF-D antibody or an antigen-binding portion thereof.

The term “anti-VEGF-D antibody” refers to an antibody that binds to VEGF-D or a biologically active fragment thereof, e.g. the mature fully-processed form, with sufficient affinity and specificity. In a certain embodiment, an anti-VEGF-D antibody is capable of binding VEGF-D with sufficient affinity such that the antibody is useful as a diagnostic and/or therapeutic agent targeting VEGF-D.

Examples of anti-VEGF-D antibodies are described in the international patent application WO2010/111746.

### Screening Methods

A further aspect of the invention relates to a method for screening a LYVE-1 antagonist capable of reducing or inhibiting lymphangiogenesis and which may be useful for preventing or treating a pathological condition associated with lymphangiogenesis (e.g. cancer and eye diseases).

For example, the screening method may measure the binding of a candidate compound to LYVE-1, or to cells or membranes bearing LYVE-1, or a fusion protein thereof by means of a label directly or indirectly associated with the candidate compound.

Alternatively, a screening method may involve measuring or, qualitatively or quantitatively, detecting ability of said candidate compound to inhibit lymphangiogenesis and efficiently prevents or treats a pathological condition associated with lymphangiogenesis.

The candidate compound may be tested for different biological properties (i) the capacity to bind to FGF2 or LYVE-1; and/or (ii) the capacity to reduce FGF2-induced lymphoangiogenesis; and/or (iii) the capacity to reduce the (Lymphatic Endothelial Cells) LEC tubulogenesis; and/or (iv) the capacity to reduce the LEC proliferation and/or (v) the capacity to reduce the LEC migration,

In one embodiment, the present invention thus relates to a method for screening a LYVE-1 antagonist comprising the steps consisting of:

a) providing a plurality of cells expressing LYVE-1 on their surface;

b) incubating said cells with a candidate compound;

c) determining whether said candidate compound binds to and inhibits LYVE-1; and

d) selecting the candidate compound that binds to and inhibits LYVE-1.

In a particular embodiment, the screening method of the invention may further comprising a step consisting of administering the candidate compound selected at step d) to an animal model (e.g. a mouse corneal lymphangiogenesis model) to validate the protective and/or therapeutic effects of said candidate compound on conditions associated with lymphangiogenesis.

In general, such screening methods involve providing appropriate cells which express LYVE-1 on their surface. In particular, a nucleic acid encoding LYVE-1 may be employed to transfect cells to thereby express the receptor of the invention. Such a transfection may be accomplished by methods well known in the art.

In a particular embodiment, said cells may be selected from the group consisting of the mammal cells reported yet to express LYVE-1 (e.g. (Lymphatic Endothelial Cells) LEC).

The screening method of the invention may be employed for determining an antagonist by contacting such cells with compounds to be screened and determining whether such compound inhibits LYVE-1.

Alternatively, the method for screening a LYVE-1 antagonist comprises the steps consisting of:

a) providing a LYVE-1 polypeptide in solution or immobilized onto a support.

b) incubating said LYVE-1 polypeptide with a candidate compound, and

c) determining whether said candidate compound binds to LYVE-1.

The support may be a solid or semi-solid support such as any suitable support comprising glass, plastic, nylon, paper, metal, polymers and the like. The support may be of various forms and sizes, such as a slide, a membrane, a bead, a column, a gel, etc.

In another embodiment, the present invention relates to a method for screening a LYVE-1 antagonist comprising the steps consisting of:

a) determining the ability of a candidate compound to inhibit the interaction between a LYVE-1 polypeptide and a FGF2 polypeptide, and

b) selecting positively the candidate compound that inhibits said interaction.

In a particular embodiment, the candidate compound has been tested for its capacity to bind LYVE-1 as previously described.

Thus, in one particular embodiment, the method for screening a LYVE-1 antagonist useful for reducing or inhibiting lymphangiogenesis comprising the steps of: (a) selecting a candidate compound by performing the methods for identifying a compound that binds to LYVE-1 as described above, (b) determining the ability of a candidate compound to inhibit the interaction between a LYVE-1 polypeptide and a FGF2 polypeptide, and (c) selecting positively the candidate compound that inhibits said interaction.

At step a), any method suitable for the screening of protein-protein interactions is suitable.

Whatever the embodiment of step a) of the screening method, the complete LYVE-1 polypeptide and the complete FGF2 polypeptide may be used as the binding partners. It should further be noted that fragments of LYVE-1 polypeptide and FGF2 polypeptide that include the site of interaction may be used as the binding partners.

The compounds that inhibit the interaction between LYVE-1 and FGF2 encompass those compounds that bind either to the LYVE-1 or to FGF2, provided that the binding of the said compounds of interest then prevent the interaction between LYVE-1 and FGF2.

Polypeptides of the invention may be produced by any technique known per se in the art, such as without limitation, any chemical, biological, genetic or enzymatic technique, either alone or in combination(s).

Knowing the amino acid sequence of the desired sequence, one skilled in the art can readily produce said polypeptides, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions.

Alternatively, the polypeptides of the invention can be synthesized by recombinant DNA techniques as is now well-known in the art. For example, these fragments can be obtained as DNA expression products after incorporation of DNA sequences encoding the desired (poly)peptide into expression vectors and introduction of such vectors into suitable eukaryotic or prokaryotic hosts that will express the desired polypeptide, from which they can be later isolated using well-known techniques.

It should be noted that said polypeptides may comprise post-translational modifications that include but are not limited to phosphorylation, acetylation, glycosylation and the like.

### Labelled Polypeptides

In one embodiment, the LYVE-1 polypeptide or the FGF2 polypeptide of the invention is labelled with a detectable molecule for the screening purposes.

Accordingly, said detectable molecule may consist of any compound or substance that is detectable by spectroscopic, photochemical, biochemical, immunochemical or chemical means. For example, useful detectable molecules include radioactive substance (including those comprising ³²P, ²⁵S, ³H, or ¹²⁵I), fluorescent dyes (including 5-bromodesosyrudin, fluorescein, acetylaminofluorene or digoxigenin), fluorescent proteins (including GFPs and YFPs), or detectable proteins or peptides (including biotin, polyhistidine tails or other antigen tags like the HA antigen, the FLAG antigen, the c-myc antigen and the DNP antigen).

According to the invention, the detectable molecule is located at, or bound to, an amino acid residue located outside the said amino acid sequence of interest, in order to minimise or prevent any artefact for the binding between said polypeptides or between the candidate compound and or any of said polypeptides.

### Test Compounds of the Invention

According to the invention, the candidate compounds may be selected from a library of compounds previously synthesised, or a library of compounds for which the structure is determined in a database, or from a library of compounds that have been synthesised de novo or natural compounds.

The candidate compound may be selected from the group of (a) proteins or peptides, (b) nucleic acids and (c) organic or chemical compounds (natural or not).

The invention will be further illustrated by the following figures and examples. However, these examples and figures should not be interpreted in any way as limiting the scope of the present invention.

### FIGURES

FIG. 1: Analysis of FGF2/LYVE-1 interaction by AlphaScreen®-based technology. (A) Detection of FGF2/LYVE-1 interaction. Top panel: assay design of the AlphaScreen® experiment. GST tagged FGF2 (FGF2-N-GST) was bound to AlphaScreen® Glutathione Donor beads and 6×His tagged LYVE-1 (LYVE-1 N-6×His) to AlphaScreen® Ni chelate acceptor beads. Bottom panel: Direct interaction between FGF2 and LYVE-1. The recombinant proteins of indicated concentrations were incubated with donor and acceptor beads for 1 h at room temperature before signal measurement. (B, C) AlphaScreen®-based competition assays. The interaction between 500 nM FGF2-N-GST and 50 nM LYVE-1 N-6×His was competed in the presence of increasing concentrations of heparin (B) and PDGF-BB (C). IC50 were determined with equation one-site competition (Graphpad PrismSoftware, San Diego, Calif.). (A through C) Results are representative experiments from three independent experiments. Results are mean values±SD (n=3).

FIG. 2: Detection of FGF2/LYVE-1 interaction using other assays. Interaction of LYVE-1 with various ligands detected by a solid-phase ligand binding assay. LYVE-1 was added to wells coated with different recombinant proteins as indicated, and incubated at 2 h at 37° C. Anti-LYVE-1, secondary peroxidase-conjugated anti-goat antibodies, and TMB substrate were used to detect bound LYVE-1. Representative experiment was done in duplicate. Error bars represent the mean±SD (n=4). Values of dissociation constants (KD) are presented in Table in the bottom.

FIG. 3: Biological function of the FGF2/LYVE-1 interaction in the endothelial cells. FGF2 stimulated migration (A), invasion (B) and proliferation (C) of LEC in the presence of soluble LYVE-1 (250 ng/ml) and/or FGF2 (20 ng/ml). Cells were incubated at 37° C. 5% CO₂ in the IncuCyte™ live-cell imaging system as described in Material and Methods. The time-course of cell migration or invasion was quantified by dynamic imaging (pictures at 2 h time interval) as percentage of scar recovery (cells migrated/invaded into the wound). The proliferation was quantified by dynamic imaging using percentage of confluence at 2 h time intervals. In the insets, diagrams of one time point are represented. Experiment was performed three times and one representative experiment is shown (n=4 for proliferation; n=6 for migration and invasion). *P<0.05,**<0.01,***P<0.001, ns, non significant vs control cells stimulated by FGF2 in the absence of LYVE-1. (D) Effect of soluble LYVE-1 on ERK phosphorylation using AlphaScreen® SureFire® phosphorylation assay. LEC were stimulated with 20 ng/ml FGF2 in the presence or absence of 250 ng/ml LYVE-1 for 5,10 and 15 min. Phosphorylated ERK1/2 in cell lysates was quantified in an AlphaScreen® SureFire® p-ERK1/2 (Thr202/Tyr204) Assay (PerkinElmer, Inc.) according to the manufacturer's instructions. Results are representative experiments from three independent experiments. Results are mean values±SD (n=3).

FIG. 4: Effect of LYVE-1 knockdown in LEC on FGF2 activity. (A) FGF2 concentration-dependent proliferation of LEC with downregulated LYVE-1. LECs were transfected for 72 h with LYVE-1 siRNA or ctr siRNA. Cell proliferation was determined by Cell Proliferation Reagent WST-1. (B-D) Effect of LYVE-1 downregulation on endothelial tube formation of LEC. LECs were transfected for 24 h with LYVE-1 siRNA or ctr siRNA and then basal and FGF2-induced capillary tube formation of LEC were performed as indicated in materials and Methods. Images of tubulogenesis processed by the Wimasis.com platform (B). Diagrams of total branching points (C) and number of loops (D). Values are the means±SD (n=3).

FIG. 5: Effect of exogenously expressed cell surface tmLYVE-1 on ¹²⁵I-FGF2 binding (A) Quantitative cell attachment assay of tmLYVE-1 CHO cells. TmLYVE-1 CHO and mock-transfected CHO cells were plated onto microtiter wells coated with HA and cell attachment was analyzed as described in Supplementary Material and Methods. The experiment was repeated two times in triplicate, values are mean±SD. (B)¹²⁵I-FGF2 binding to low- affinity sites in the tmLYVE-1 CHO clone. TmLYVE-1 CHO and mock-transfected cells were incubated with 10 ng/ml ¹²⁵I-FGF2 in the presence or absence of 100 ng/ml heparin. After 2 h of incubation at 4° C., the radioactivity associated with HSPGs was measured as indicated in Materials and Methods. Data are expressed as a specific activity (¹²⁵I-FGF2 ng/10⁶ cells). The binding of the figure depicts representative experiment done in duplicates (data points as mean±SD, n=3). (C) ¹²⁵I-FGF2 binding to high-affinity sites in tmLYVE-1 CHO-R3. TmLYVE-1 CHO-R3 and mock-transfected cells were incubated with increased concentrations of ¹²⁵I-FGF2. After 2 h of incubation at 4° C., the radioactivity associated with FGFR was measured as indicated in Materials and Methods. Data are expressed as a specific activity (¹²⁵I-FGF2 ng/10⁶ cells). The binding of the figure depicts representative experiments done in duplicates (data points as mean±SD, n=3).

FIG. 6: Effect of LYVE-1 on FGF2-induced lymphangiogenesis in vivo. Corneal lymphangiogenesis following implantation of pellets containing FGF-2 alone or FGF-2 and LYVE-1. Quantitative analysis of corneal lymphangiogenesis induced by FGF2 or FGF2/LYVE-1 containing pellets (n_{FGF2}=21, n_{FGF2/LYVE-1}=20, p=0.0008). Data represent mean±SD

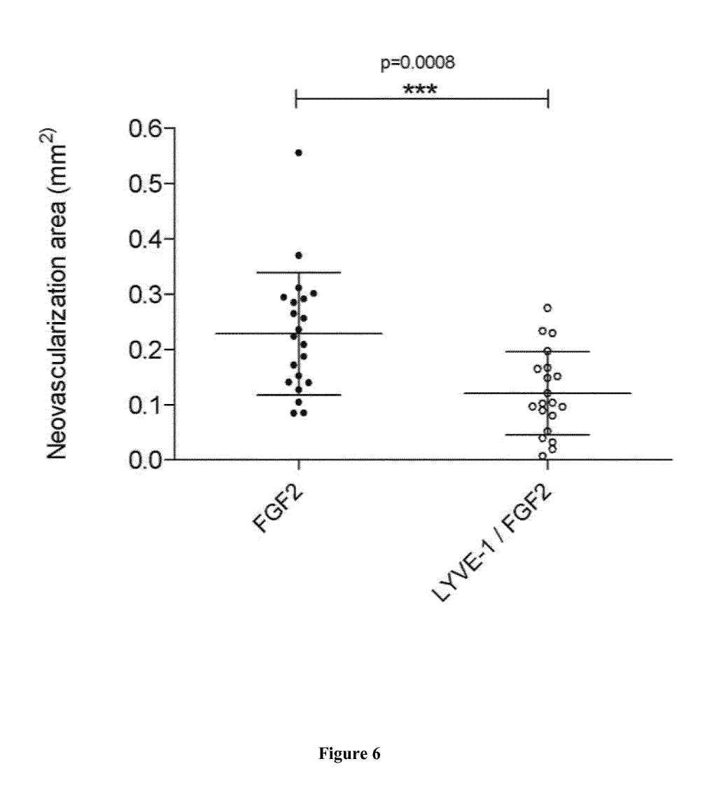

FIG. 7: Effect of FGF2 on endogenous LYVE-1 expression in endothelial cells. (A) Endogenous LYVE-1 expression in FGF2-stimulated endothelial cells. LECs and HUVECs were stimulated with 10 ng/ml FGF2 in a time-dependent manner 24-48-72 h. Endogenous LYVE-1 was measured by qPCR. Results are expressed in fold change of LYVE-1 mRNA in FGF2-stimulated cells vs. non stimulated cells on each time point using the GAPDH reference gene. Reversion of the TNFβ-dependent downregulation of LYVE-1 by FGF2 in LECs (B) and HUVECs (C). LECs and HUVECs were stimulated with 10 ng/ml FGF2 for 24-48-72 h and 10 ng/ml TNFβ was added to the culture medium. Endogenous LYVE-1 was measured by qPCR as above. Values obtained by qPCR are the means±SD (n=3).

FIG. 8: Biological function of the FGF2/LYVE-1 interaction in the endothelial cells. (A) VEGF-C stimulated migration of LEC in the presence of soluble LYVE-1 (250 ng/ml) and/or VEGF-C(20 ng/ml). Cells were incubated at 37° C. 5% CO₂ in the IncuCyte™ live-cell imaging system. The time-course of cell migration was quantified by dynamic imaging (pictures at 2 h time interval) as percentage of scar recovery (cells migrated/invaded into the wound). Experiment was performed three times and one representative experiment is shown (n=4). (B) Apoptosis of LEC in the presence of exogenous soluble LYVE-1. Serum starvation and increasing concentration of LYVE-1 were assayed for apoptosis induction by quantifying the activation of caspase-3/7. GM, growth medium. Values are mean±SD of at least three independent experiments (n=4). *P<0.05, ***P<0.001, ns, non significant vs control cells stimulated by FGF2 in the absence of LYVE-1.

FIG. 9: Effect of LYVE-1 downregulation. (A) Validation of LYVE-1 knockdown in LEC. LYVE-1 mRNA expression in LYVE-1 siRNA-transfected (LYVE-1 siRNA) and nontarget siRNA-transfected cells (ctr siRNA) was determined by qPCR. Results are expressed in fold change of LYVE-1 in LYVE-1 siRNA cells vs. ctr siRNA LEC using GAPDH as the reference gene. In the inset: Western blot of immunoprecipitated with protein A beads total LYVE-1 protein. Non-transfected and ctr siRNA LEC (1,2 lane) expressed much higher levels of LYVE-1 protein compared with LYVE-1 siRNA transfected LEC (3 lane). (B) VEGF-stimulated proliferation of LEC with down-regulated LYVE-1. (C) FGF2-stimulated proliferation of BEC in the presence of LYVE-1 siRNA. Cells were grown for 72 h and cell proliferation was determined by Cell Proliferation Reagent WST-1. Results are expressed as percentages of induction; the highest value being set as 100%. Results are represented as the mean values±SD (n=4).

### EXAMPLE

### Material & Methods

Cell Lines:

Human Dermal Lymphatic Endothelial Cells (LEC, Promocell, France) and Human Dermal Blood Endothelial cells (BEC, Promocell) were grown in EGM MV2 (Promocell) and in EGM MV (Promocell), respectively, supplemented with 5% fetal calf serum (FCS) and with growth factors according to the manufacturer's instructions. Human umbilical vein endothelial cells (HUVEC, Lonza, France) were maintained in EBM-2 (Lonza) supplemented with EGM-2 SingleQuots (Lonza), which contains 2% FCS. CHO and CHO FGFR3 cells (kindly provided by M. Presta) were cultured in DMEM/F12 containing 4.5 g/l glucose (Gibco, France), supplemented with 10% FBS, 2 mM glutamine and penicillin/streptomycin. All cells were grown at 37° C. in a 5% CO₂ atmosphere.

Amplified Luminescence Proximity Homogeneous Assays (AlphaScreen®):

Reaction mixtures were prepared in 20 μl final volume in 384-well plates. Firstly, 5 μl of each prepared dilutions of FGF2-N-GST and LYVE-1 N-6×His in the AlphaScreen® reaction buffer (50 mM Hepes pH 7.4, 150 mM NaCl, 5 mM MgCl₂, 1 mM DTT, 0.1% BSA and 0.05% Tween-20) were incubated together for 30 min at room temperature. After, 5 μl of AlphaScreen® Glutathione Donor beads and 5 μl of Ni Chelate Acceptor beads (PerkinElmer, Inc., 25 μg/ml final concentration) were added to the mix and the plate was further incubated for 1 h at room temperature before signal measurement. When a competition assay was performed, 5 μl of a competitor (at various concentrations) were added to the mix for 30 min at room temperature before incubation with the beads. Plates were read on EnVision®2103 Multilabel Plate Reader (PerkinElmer, Inc.) equipped with AlphaScreen® optical detection module.

¹²⁵I-FGF2 Binding Assay:

10 μg of recombinant FGF2 was labeled with ¹²⁵I-Na (1mCi) using Iodogen precoated tubes (Pierce Biotechnology, USA) according to the manufacturer's indications and as previously described⁽¹¹⁾. The specific activity of ¹²⁵I-FGF2 was 80,000 to 100,000 cpm/ng. Binding experiments to high- and low-affinity sites were performed essentially as described by Roghani and Moscatelli¹¹. CHO cells (2.5×10⁵/well) were seeded onto 6-well dishes coated with 0.15% gelatin, and grown for 2 days. Cells were washed twice with ice-cold phosphate buffer saline (PBS) before binding and incubated with indicated concentrations of ¹²⁵I-FGF2 in DMEM containing 20 mM HEPES (pH 7.4), 0.15% gelatin for 2 hours at 4° C. Then, after a PBS wash, cells were washed twice with 2 M NaCl in ice-cold 20 mM HEPES buffer (pH 7.4) to remove ¹²⁵I-FGF2 bound to low-affinity heparan sulfate proteoglycan (HSPGs) or twice with 2 M NaCl in ice-cold 20 mM sodium acetate (pH 4.0) to remove ¹²⁵I-FGF2 bound to high-affinity FGF receptor (FGFRs). Nonspecific binding was measured in the presence of a 100-fold molar excess of unlabeled FGF2 or 1 μg/ml protamine sulfate and subtracted from all values. Bound ¹²⁵I-FGF2 was quantified using a Kontron MR 250 gamma counter.

Small Interfering RNA (siRNA) Knockdown Experiments:

LEC were plated at a density of 8×10⁴ cells per well in six-well plates. siRNA against human LYVE-1, ON-TARGETplus SMARTpool, ON-TARGETplus siRNA-Human XLKD1 no. J-020129 and nontargeting siRNA pool were from Dharmacon, USA. Transfection was performed using lipofectamine RNAiMAX (Invitrogen, Carlsbad, USA) according to the manufacturer's instructions with a final siRNA concentration of 10-83.5 nM for 48-96 h. siRNA-transfected cells were then used for RNA and protein extraction, tubulogenesis or proliferation assays.

Cross-Linking of FGF2 and LYVE-1:

The amine-specific homobifunctional cross-linker bis(sulfosuccinimidyl) suberate (BS3; Pierce Biotechnology, USA) was used to cross-link FGF2 and LYVE-1 according to the protocol modified from Perollet et al⁽¹²⁾. To this end, the recombinant FGF2-C-his was labeled with IRDye® 800CW Protein Labeling Kit-Low MW (LI-COR® Biosciences, Lincoln, Nebr.) according to the manufacture's instruction. The labeled FGF2-IRDye conjugate was characterized with a dye to protein ratio 3.3. The labeled FGF2 was incubated with increasing concentrations of LYVE-1 for 1 h at room temperature and after incubated for 30 min with 1 mM freshly prepared BS3 solution. Each reaction mixture was quenched with 1 M Tris HCl at pH 8.0. Cross-linked samples were analyzed by 10% SDS-PAGE.

Tubulogenesis of LYVE-1 siRNA LEC:

LEC were plated at 1×10⁵ cells in 24-well plates and incubated with 83.5 nM LYVE-1 siRNA for 24 h. Cells were then starved overnight in endothelial basal medium (EBM). The next day, 10,000 cells were added on IBIDI μ-slide coated with 7 μg/μL (10 μL) reduced matrigel in EBM with or without 50 ng/ml FGF2. LECs were incubated for 12-24 h at 37° C. in a 5% CO₂ humidified atmosphere and the effects on endothelial tube formation were estimated. Four digitized pictures were made per well and the images were processed with the Wimasis.com platform (Wimasis.com) to determine the total branching points and the number of loops.

Cell Proliferation:

LEC (3000 cells/well) were plated in a 96-well plate and allowed to adhere overnight. Then complete medium was replaced by EBM supplemented with 0.2% FBS for 7 h followed by treatment with 20 ng/ml of recombinant FGF2 in the presence or absence of 250 ng/ml LYVE-1. For time-course experiments to measure cell proliferation IncuCyte™ technology (Essen Bioscience) was used. Growth curves were constructed by imaging plates where the growth curves were built from confluence measurements acquired during round-the-clock kinetic imaging. Alternatively, standard colorimetric proliferation assay with WST-1 (Roche) was used. As control, we carried out the tests in the absence of FGF2. For siRNA proliferation experiments, siRNA LYVE-1 transfection was performed 24 h before starvation.

Cell Migration and Invasion Assays:

LEC migration and invasion assays were carried out using IncuCyte™ (Essen Bioscience). 3×10⁴ cells/well were plated in 96-well ImageLock plates (Essen Bioscience) precoated either for 6 h with 50 μg/ml of reduced matrigel (BD Biosciences) for invasion or with 10 μg/ml of fibronectin (Sigma) for migration. At 90-100% of confluence the plates were scratched with 96-Well WoundMaker™ (Essen Bioscience) and complete medium was replaced by serum-free medium containing or not recombinant proteins. For invasion assay 150 μg/ml of reduced matrigel was added on each well. Migration/invasion was detected by IncuCyte™ scaning one image per well, every two hours for 30 hours. Time-course of cell migration/invasion was quantified using percentage of recovery scar (cells migrated/invaded into the wound) at 2 h time intervals.

Solid-Phase Ligand Binding Assay:

Solid-phase ligand binding assay was performed as described previously with minor modifications⁽¹³⁾. 96-well plates (Immulon 2 HB microtiter wells, ThermoFisher Scientific) were coated with 500 nM of recombinant proteins in PBS for 2 h at 37° C. The wells were washed 4 times with a buffer (10 mM Tris, pH 7.4, 0.15M NaCl and 0.1% Triton X-100) and blocked with 1% BSA in PBS at room temperature for 2 h. LYVE-1 recombinant protein in 0.1% BSA was added on coated wells and incubated for 2 h at 37° C. Unbound LYVE-1 was extensively washed. The bound molecules were incubated with a goat anti-LYVE-1 antibody (1:500) for 1 h at room temperature. As secondary antibody peroxidase-conjugated goat anti-rabbit IgG (Dako, USA) were used. TMB liquid substrate (Sigma) was used for revelation. Absorbance was measured at 450 nm in a microplate reader. Background absorbance in the wells coated with BSA was deducted from the values obtained.

Statistics:

Data are presented as mean±SD. Statistical analyses were performed using unpaired Student's t-test. *, P<0.05; **, P<0.01; ***, P<0.001.

Materials:

Heparin from porcine intestinal mucosa, hyaluronan from rooster comb and human fibronectin were purchased from Sigma, recombinant PDGF-BB or VEGFA were from Reliatech GmbH, soluble extracellular part of recombinant human LYVE-1 (tagged with 6×His), recombinant human TNFβ, recombinant FGF2 were from R&D Systems. Recombinant VEGF-C was from Reliatch GmbH or R&D Systems. Rabbit anti-FGF2 antibodies were from Santa-Cruz Ltd, monoclonal anti-FGF2 antibodies were from Epitomics, PGNaseF was from Sigma, goat anti-human LYVE-1 antibodies were from R&D Systems, and rabbit anti-human LYVE-1 antibodies from Reliatech GmbH, Germany, rabbit anti-mouse LYVE-1 antibodies from AngioBio or Abcam.

Plasmid Constructions and Recombinant Protein Production:

Full-length human LYVE-1 cDNA was amplified by PCR from pCMV6-XL5 vector containing the human LYVE-1 cDNA (Origene, USA) and a 1019 bp amplicon was subcloned into the pcDNA3.1/Hygro(+) vector (Invitrogen, Carlsbad, USA) using HindIII and XbaI as restriction sites. The sequence was confirmed by sequencing. For generation of cell lines expressing exogenous LYVE-1, CHO cells were stably transfected with the LYVE-1 pcDNA3.1/Hygro(+) plasmid using Lipofectamine-2000 (Invitrogen). After 2-3 weeks of selection with 800 μg/ml hygromycine (Invitrogen), single colonies were picked and expanded. The selected clones were screened by QPCR for LYVE-1 expression. The cDNA encoding the 18 kDa FGF2 form was amplified by PCR using FGF2-specific primers that were containing the attB adapter sequence and was cloned in the pDONR201 vector (Invitrogen) using the GATEWAY BP-reaction system (Invitrogen). The positive entry clones were sequence-verified and used to create the final GATEWAY-expression constructs by LR-cloning (Invitrogen) in GATEWAY modified pGEX-2TK (N-GST) or in pDEST42 (C-His, Invitrogen). Expression constructs were transfected into BL21(DE3)pLysS. Protein expression was induced at 37° C. for 2-3 h by addition of 1 mM isopropyl-β-Dithiogalactopyranoside to exponentially growing cells. Tagged FGF2 recombinant proteins were purified by affinity chromatography using GST-HiTrap or Ni²⁺ HiTrap column (GE Healthcare, USA) according to the manufacturer's instructions and characterized as described by Patry et al. 1994⁽²¹⁾. The molecular mass and purity of the protein were analyzed using SDS-PAGE and Western blotting with anti-FGF2 antibodies after dialysis against 50 mM HEPES buffer, pH 7.4.

Deglycosylation:

LYVE-1 glycosylation was evaluated following PGNaseF treatment. Briefly 2 ug of LYVE-1 were either treated or not with PGNaseF as recommended by the manufacturer (Pierce). Proteins were then resolved by SDS-PAGE (10%) and immunobloted using anti-LYVE-1 antibodies. 150 nM of LYVE-1 control or PGNaseF-treated was assayed with 500 nM FGF2 as previously described.

Apoptosis Assay:

Apoptosis was evaluated by measuring caspase 3/7 activities with the Apo-one homogeneous assay kit, according to manufacturer instructions (Promega, France). LECs were seeded in 96-well plates at a concentration of 5×10³ cells/well and allowed to adhere overnight. Complete medium was replaced by serum-free medium containing or not recombinant proteins. Apoptosis was measured after 48 h using Fluostar OPTIMA fluorometer (BMG Labtech).

Cell Signaling:

LEC were stimulated with 20 ng/ml FGF2 in the presence or absence of 250 ng/ml LYVE-1 for 5-15 min. After stimulation the medium was removed and the cells were washed with PBS to eliminate all traces of phenol red. Cellular lysates were prepared with 1× lysis buffer for AlphaScreen® SureFire® Assay (PerkinElmer, Inc.) and transfer to a 384-well Proxiplate for assay. AlphaScreen SureFire technology allows the detection of phosphorylated proteins in cellular lysates. Activated extracellular regulated kinase (ERK1/2) in cell lysates was quantified in an AlphaScreen® SureFire® p-ERK1/2 (Thr202/Tyr204) Assay (PerkinElmer, Inc.) according to the manufacturer's instructions. Plates were read on an EnVision® 2103 Multilabel Plate Reader (PerkinElmer, Inc.).

Quantitative Real-Time PCR (qPCR):

RNAs from LECs or from 25 mg of frozen mice tissue were extracted using the RNeasy Mini Kit (Qiagen, France) and quantified by OD 260 nm with NanoDrop Spectrophotometer ND-1000. Total RNAs (500 ng) were reverse-transcribed by random hexamer priming using a High-Capacity cDNA Reverse Transcription Kit (Applied Biosystems, USA). Obtained cDNAs were analysed by quantitative PCR GenAmp 5700 system and ABsolute™ QPCR SYBR Green Mix (ABgene, USA) were used to quantify the cDNA. cDNAs in triplicate were amplified with a specific primer to human/murine LYVE-1, validated for efficiency and specificity of amplification. Relative quantification was normalized to human housekeeping gene GAPDH or mouse β-actin, respectively.

Immunoprecipitation:

Cells in 6-well plates were lysed at 4° C. with CelLytic™ Cell Lysis Reagent (Sigma, France) and Protease Inhibitor Cocktail (Roche). The total protein concentration was determined using the Bio-Rad Protein Assay Kit (Bio-Rad, France). 40 μg of total protein were added to the 50 μl Dynabeads protein A (Invitrogen) with bound rabbit anti-LYVE-1 antibodies and incubated for 1 h at 4° C. in rotation. After wash in PBS, elution was performed by boiling the beads for 5 min in a loading dye SDS-PAGE buffer. The proteins were separated on 10% SDS-PAGE gel and transferred to a nitrocellulose membrane (Amersham), probed with primary antibodies goat anti-LYVE-1 and revealed with a secondary antibody coupled to IRDye 800CW using an Odyssey Infrared Imaging System (LICOR®).

For co-immunoprecipitation of FGF2/LYVE-1 complex in solution, FGF2-N-His (MW 24 kDa) and LYVE-1-His were used. Monoclonal FGF2 antibody and polyclonal LYVE-1 antibody were bound separately to Dynabeads protein G (Invitrogen) according to the manufacturer's protocol. To each antibody/protein G complex, the FGF2/LYVE-1 (500/150 nM) complex preincubated overnight at 4° C. was added. After washing, the proteins were eluted by boiling in loading buffer and then processed for Western blot analysis.

Quantitative Cell Attachment Assay:

96-well plates (Immulon 2 HB microtiter wells ThermoFisher Scientific, USA) were coated with HA rooster comb, 400 μg/well for 2 h at 37° C. and blocked with 1% BSA 1 h at room temperature. CHO cells were resuspended in serum-free medium supplemented with 0.5% BSA, seeded in wells (5×10⁴ cells/wells) and allowed to adhere for 1 h. Non-adherent cells were removed by two washes with PBS. Adherent cells were fixed with 30% methanol+10% acetic acid and stained with 0.1% Coomassie blue in 30% methanol+10% acetic acid. After wash with PBS, stained cells were solubilized by 1% SDS and to quantify cell adhesion, the absorbance was measured at 620 nm. Background absorbance in the wells coated with BSA was deducted from the values obtained.

Corneal Micropocket Assay:

The mouse corneal micropocket assay was performed as described previously with modifications (Rogers at al., 2007⁽²²⁾). Animals were deeply anaesthetized and a corneal micropocket was prepared towards the limbus. Sucralfate pellets containing a combination of 80 ng recombinant human FGF-2 (rhFGF-2) or 200 ng rhVEGF-C respectively and 1 μg recombinant human LYVE-1 were coated with hydron and implanted into the corneal stroma. Control pellets contained 80 ng rhFGF-2 or 200 ng rhVEGF-C respectively. The rhFGF-2 pellets (or rhVEGF-C pellets) were created by mixing four μg rhFGF-2 (or 10 μg rhVEGF-C) in PBS with 2.25 mg sucralfate. The mixture then was placed in a vacuum centrifuge until dry. For pellets containing rhFGF-2 (or rhVEGF-C) and LYVE-1 four μg rhFGF-2 (or 10 μg rhVEGF-C) in PBS were mixed with 2.25 mg sucralfate and centrifuged until dry. Then 50 μg LYVE-1 in PBS were added and the solution was again centrifuged until dry. Finally 12% hydron (poly-HEMA) in ethanol was added and the mixture was pipetted subsequently onto a nylonmesh (7×7 squares) with an approximate pore size of 400×400 μm. The suspension was allowed to dry at room temperature and afterwards the fibers of the nylon mesh were pulled apart under a stereomicroscope. Uniform pellets were picked out to ensure that each pellet contained the right amount of growth factor. Antibiotic ointment was applied to each operated eye postoperatively. On postoperative day seven the mice were euthanized, the cornea with limbus was excised and the lymphangiogenic response was measured.

For immunohistochemistry, corneal tissue was carefully excised along the limbus and rinsed in PBS. Corneas were fixed in acetone for 20 min at room temperature and washed with PBS. After a blocking 1 h with 2% BSA in PBS, corneal whole mounts were incubated with anti-mouse LYVE-1 antibody diluted in PBS (1:200) overnight at 4° C. Next day corneal tissue was rinsed in PBS and incubated in PBS with the secondary antibody (goat anti-rabbit cy3) for 45 min at room temperature in the dark to detect binding of the primary antibody. Finally corneal whole mounts were washed in PBS and transferred to Superfrost®-slides. The tissue was coverslipped with DAKO® fluorescent mounting medium and stored at 4° C. in the dark prior to analysis with a fluorescence microscope.

For morphometrical analysis and semiautomatic quantification of corneal lymphangiogenesis stained whole mounts were analyzed with a fluorescence microscope: digital pictures were taken with a 12-bit monochrome CCD camera. Each whole mount picture was assembled out of at least nine pictures (depending on the size of the cornea) taken at 100× magnification. The area covered with lymphatic vessels excluding the limbal vessels was detected with an algorithm established in the image analysis program cell̂F: prior to analysis grey value images of the whole mount pictures were modified by several filters (Bock et al. 2008⁽²³⁾). LYVE-1⁺ stained lymphatic vessels were detected by threshold setting including the bright vessels and excluding the dark background as described previously⁽²³⁾.

Mouse Dermal Explants:

Male BALB/c aged 7 weeks were euthanized by cervical dislocation, and the ears were removed, incubated in penicillin/streptomycin on ice for 30 min, and then split into dorsal and ventral halves and floated split-side (dermis) down in RPMI 1640, 10% fetal calf serum, penicillin (1 units/ml), streptomycin (50 mg/ml), and 2 mM L-glutamine. Explants were cultured in a humidified atmosphere at 37° C. in 5% CO2 in the presence of recombinant human TNFβ and/or FGF2 at 200 ng/ml for 48 h.

Immunofluorescent Staining of Cells and Tissues:

For single/double immunofluorescent staining, cells grown on glass coverslips in 24-wells were fixed in 4% paraformaldehyde (Santa Cruz Biotechnology) and rinsed with PBS. After 1 h blocking with 5% BSA, appropriate primary antibodies were applied in 1% BSA and incubated at 25° C. for 1 h followed by wash and further incubation with the appropriate Alexa Fluor conjugated secondary antibodies (1:750, Molecular Probes). For single staining nuclei were counterstained with DAPI (4,6 diamidino-2-phenylindole, 1:2000). Cells were mounted in Prolong (Invitrogen), and viewed on a confocal microscope (LEICA, SP5). For whole-mount staining, tissue was fixed overnight at 4° C. in 4% paraformaldehyde, permeabilized in PBS-Triton X-100 (0.1% v/v), blocked in 1× blocking reagent (Roche), and incubated overnight at 4° C. with the appropriate primary antibodies. After wash, tissue was incubated with Alexa Fluor conjugated secondary antibodies (Molecular Probes) for 2 h in the dark at 25° C. and then mounted in VECTASHIELD (DAKO) and viewed on a confocal microscope (LEICA, SP5).

FGF2−/− Mice:

FGF2−/− mice tissues were kindly provided by A. Dubrac and H. Prats (INSERM, Toulouse, France). Mice were housed and treated in the animal facility of Bordeaux 1 University (“Animalerie Mutualisée Bordeaux I”). All animal procedures were done according to institutional guidelines and were approved by the INSERM institutional animal care committee.

Results

Identification and Characterization of the Interaction Between FGF2 and LYVE-1:

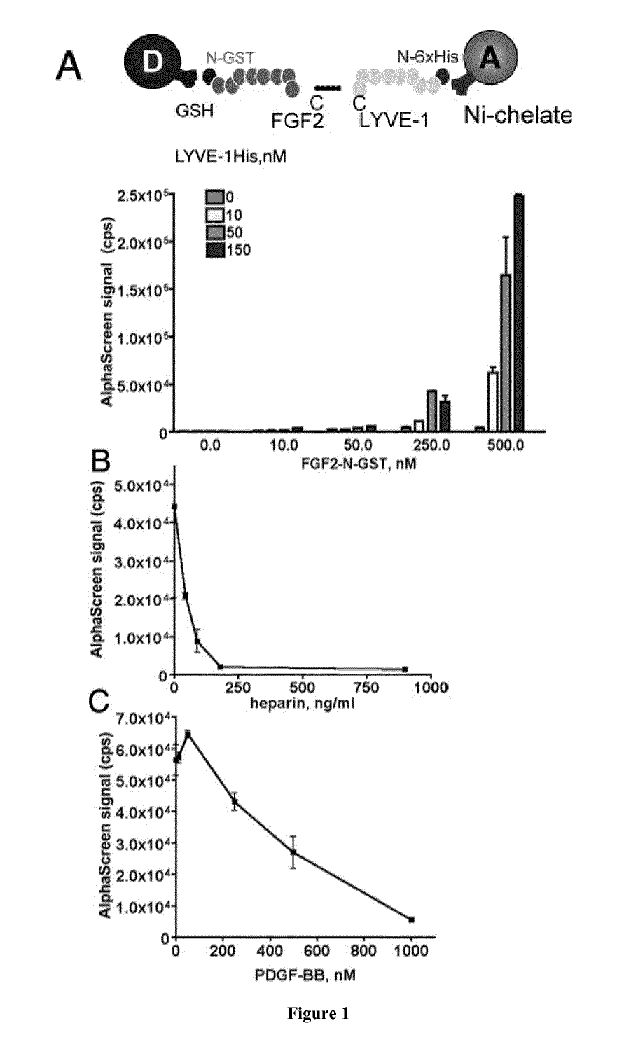

The inventors used an amplified luminescence proximity homogeneous assay, AlphaScreen® to detect and measure the interaction between FGF2 and LYVE-1. This assay allows for the characterization of the interaction between two biological partners that are bound through functional groups to acceptor and donor beads¹⁴. To configure an AlphaScreen® assay to monitor the interaction between FGF2 and LYVE-1, recombinant tagged proteins for FGF2 and LYVE-1 were used. FGF2 fused to GST was expressed in E. coli, purified using glutathione-Sepharose affinity chromatography. The purity (SDS-PAGE and Western blotting,) and the biological activity of FGF2 to stimulate endothelial cell proliferation were verified. In addition, a commercial preparation of soluble extracellular part of LYVE-1 was used. In the present assay, GST tagged FGF2 was bound to AlphaScreen® Glutathione Donor beads and 6×His tagged LYVE-1 to Nickel Chelate acceptor beads. The AlphaScreen® assay was first carried out using increasing amounts of FGF2 (10-500 nM) and LYVE-1 (10-150 nM) (FIG. 1A). A maximal AlphaScreen® signal reflecting the direct interaction between FGF2 and LYVE-1 was reached at concentrations of 500 nM FGF2 and 150 nM LYVE-1. This suggests a FGF2/LYVE-1 interaction of 3:1. They next performed competition assays in the presence of increasing concentrations of PDGF-BB or heparin. Both molecules competed with the FGF2/LYVE-1 interaction with IC₅₀ 27.2 ng/ml for heparin and 360 nM for PDGF-BB (FIGS. 1B and 1C).

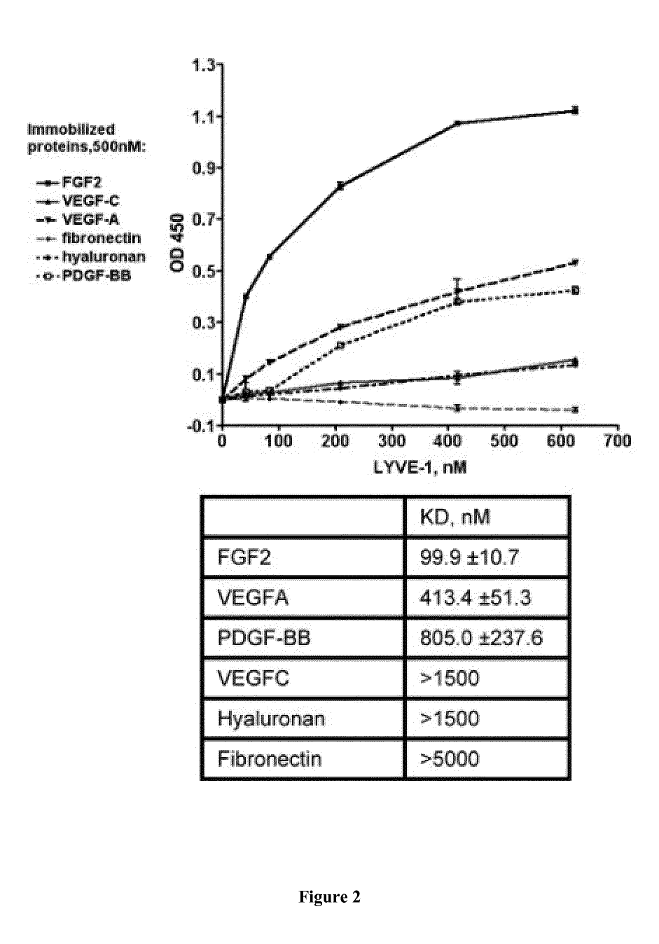

In addition to the AlphaScreen® proximity assay, they further analyzed the FGF2/LYVE-1 interaction by another assay measuring the interaction in solution or by solid phase binding assay. As shown in FIG. 2A, LYVE-1 interacted with immobilized FGF2 in a solid-phase ligand-binding assay¹³. The dissociation constant is lower than that for VEGFA, PDGF-BB, VEGFC, fibronectin and HA. This indicates that LYVE-1 interacted with FGF2 with a higher affinity than with the other potential binding molecules tested. They finally performed an interaction assay to document the FGF2/LYVE-1 interaction in solution. To this end, 10 ng of labeled FGF2-IRDye800CW were incubated with increasing amounts of LYVE-1 (100-1000 ng/well, molar ratio of FGF2/LYVE-1 1/4.4-44) in the absence or presence of unlabeled FGF2 and cross-linked in solution using BS3. Cross-linked FGF2/LYVE-1 oligomers were detected in the presence of increasing concentrations of LYVE-1.

They then performed co-immunoprecipitation experiments using both anti-FGF2 and anti-LYVE-1 antibodies. These experiments revealed that FGF2/LYVE-1 complexes could be co-immunoprecipitated using either by anti-FGF2 or anti-LYVE-1 antibodies. Taken together, these data demonstrate that FGF2 directly interacts with LYVE-1 in vitro.

They next investigated whether glycosylation is important for the FGF2/LYVE-1 interaction. Glycosylation was done using PGNaseF and the interaction of the deglycosylated product with FGF2 was investigated using ALPHA screen. Deglycosylation led to a drastic decrease in the ALPHAscreen signal. This indicates that glycosylation is important for the FGF2/LYVE-1 interaction.

Role of the FGF2/LYVE-1 Interaction in LEC:

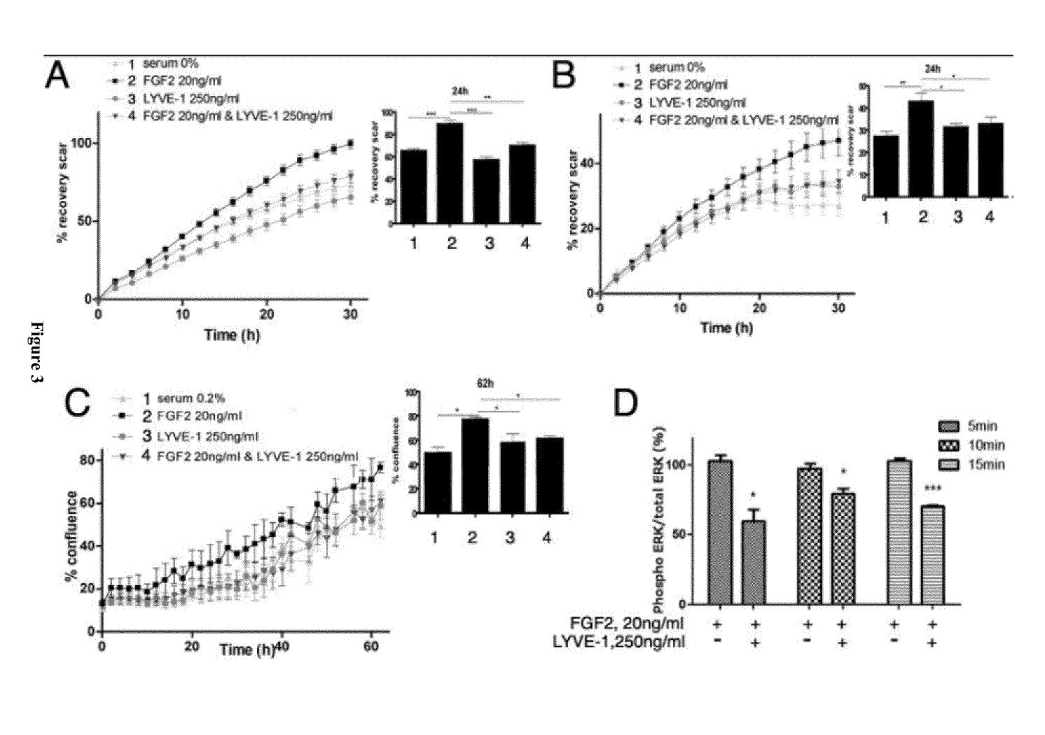

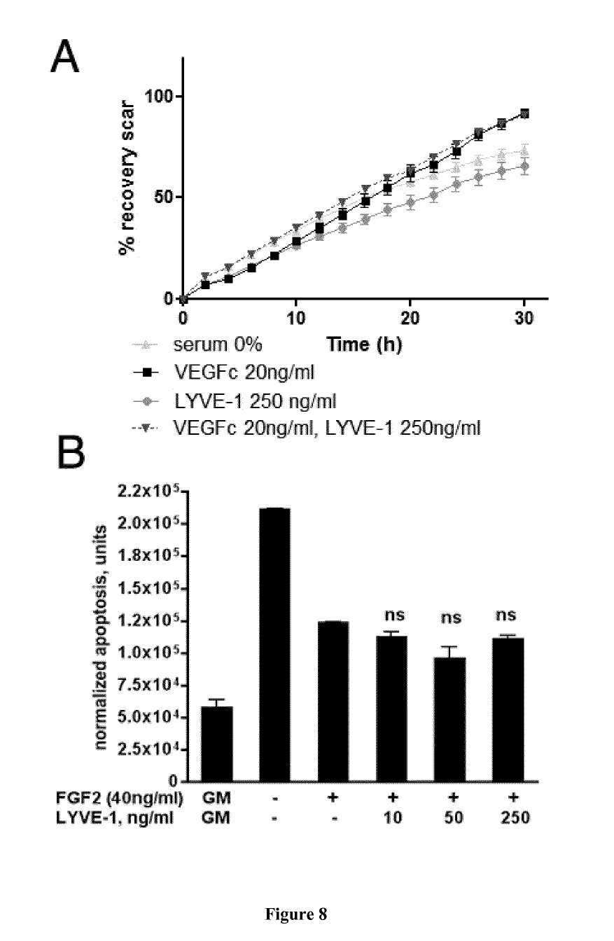

The inventors first examined whether the soluble extracellular part of LYVE-1 (sLYVE-1) inhibits the FGF2-mediated effects in LECs. To this end, they conducted both migration and invasion assays using IncuCyte™ technology. As shown in FIGS. 3A and B, sLYVE-1 (250 ng/ml) inhibited FGF2 induced migration and invasion in a time dependent manner. These experiments were confirmed by Boyden chamber migration and invasion assays. Moreover, VEGF-C induced migration was not inhibited by sLYVE-1 (FIG. 8A). In addition, they also observed LYVE-1-dependent inhibition of FGF2-induced proliferation in the IncuCyte™ assay but not of apoptosis (FIG. 3C and FIG. 8B). They next investigated whether LYVE-1 exhibited inhibitory effect on FGF2 signalling in LEC. It is well known that FGF2 induces the phosphorylation of extracellular signal-regulated kinases 1/2 (ERK1/2) by ERK kinase¹⁵.

Therefore they examined whether FGF2-stimulated ERK phosphorylation was altered by LYVE-1 in LEC. For this purpose, they used the AlphaScreen® SureFire® p-ERK1/2 (Thr202/Tyr204) Assay (PerkinElmer, Inc.), which allows the quantification of ERK phosphorylation. As shown in FIG. 3D, they observed a significant inhibition of ERK phosphorylation in LEC. Thus, these results indicate that sLYVE-1 acts on endothelial cell migration, invasion and on cell proliferation but not on apoptosis. This is in agreement with the inhibition of ERK phosphorylation.

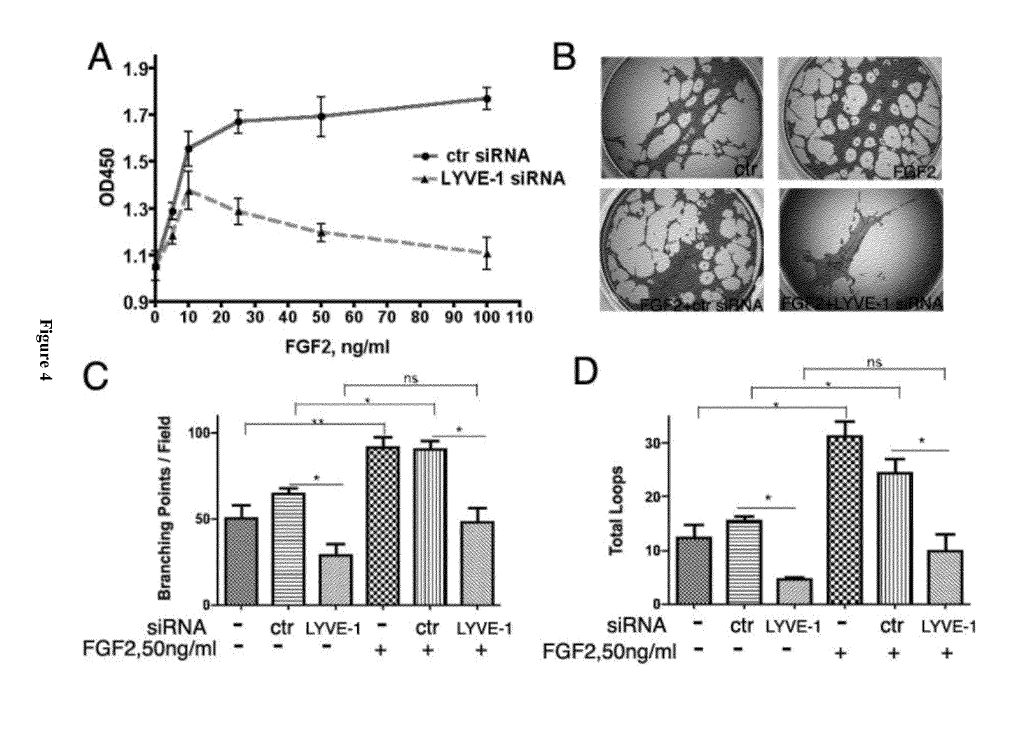

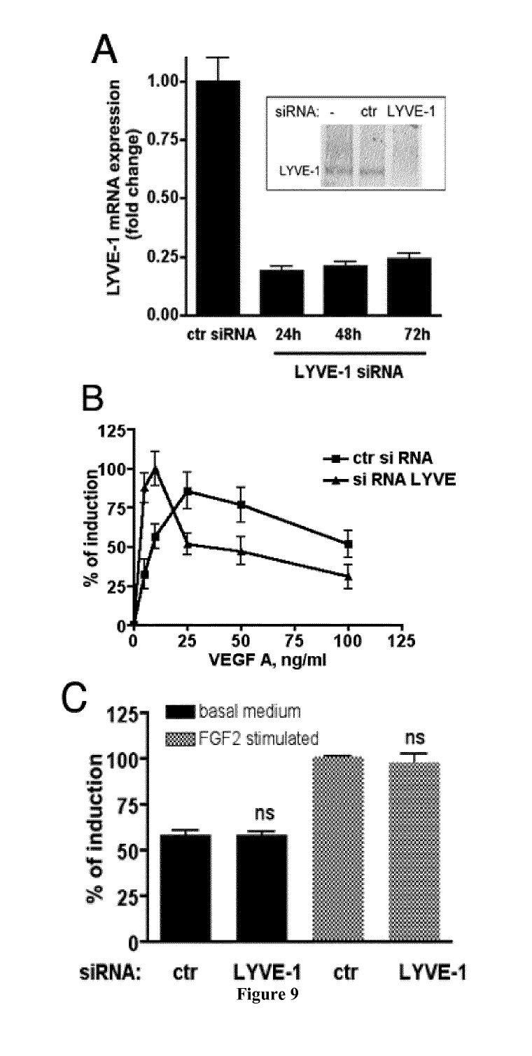

In a second approach, they investigated whether the down-regulation of membrane-bound LYVE-1 in LEC altered FGF2 activity. Knock-down of LYVE-1 in LEC was achieved using a pool of four LYVE-1 siRNAs or a selected LYVE-1 siRNA (ON-TARGETplus siRNA-Human XLKD1 no. J-020129) and resulted in the inhibition of LYVE-1 mRNA and protein (FIG. 9A). They observed an inhibition of FGF2-stimulated LEC proliferation after knock-down of membrane LYVE-1 (FIG. 4A). As a control, they attenuated LYVE-1 expression in VEGF-stimulated LECs. No significant inhibition of LEC proliferation was seen under this condition (FIG. 9B). To ascertain the specificity of LYVE-1 siRNA for lymphatic cells, FGF2-stimulated blood vascular endothelial cells (BEC) were incubated with LYVE-1 siRNA. No effect of LYVE-1 siRNA was observed under these conditions (FIG. 9C). This indicated the specificity of LYVE-1 siRNA for LECs that express LYVE-1. Finally, they observed an inhibition of FGF2-induced tubulogenesis when LYVE-1 was silenced (FIG. 4B-D). These data demonstrate that attenuation of LYVE-1 expression results in an inhibition of biological processes stimulated by FGF2.

Cell-Based Characterization of the FGF2-LYVE-1 Interaction:

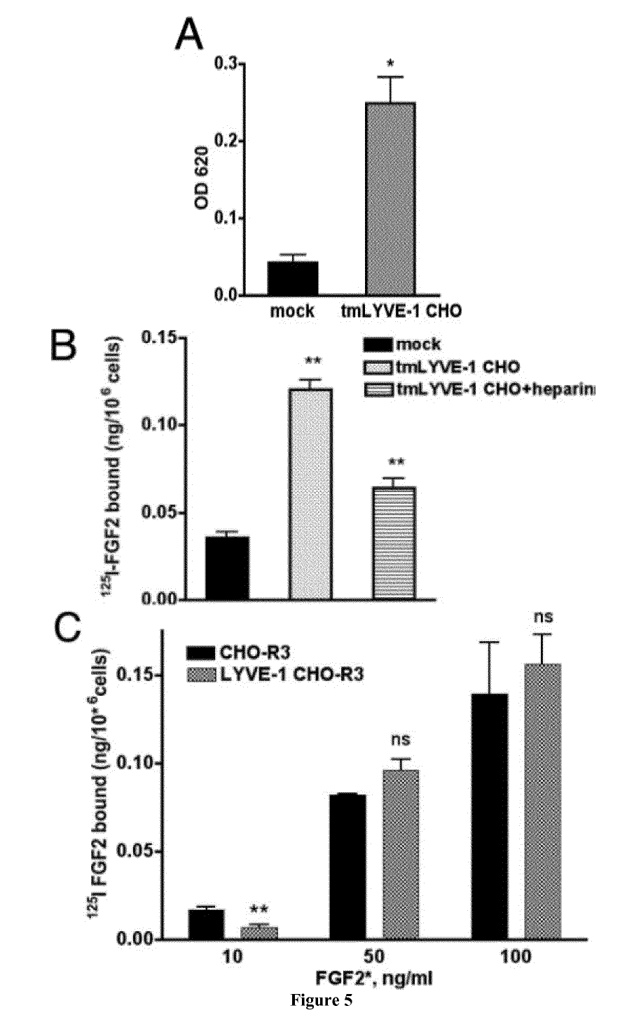

The inventors next examined whether the interaction of FGF2 and LYVE-1 occurs in cell culture systems. To this end, they cloned the full-length transmembrane LYVE-1 (tmLYVE-1) cDNA in the pcDNA3.1 vector, and stably transfected CHO cells that lack endogenous LYVE-1. After selection, several clones were obtained and analyzed for LYVE-1 mRNA expression using qPCR and for cell surface localization of LYVE-1 using immunofluorescence with an antibody specific for human LYVE-1. The isolated clones were first verified for their capacity to bind HA, a ligand of LYVE-1. Quantitative cell attachment assay demonstrated that the LYVE-1 expressing cells were able to bind to immobilized HA 2 fold more than mock-transfected cells lacking LYVE-1 (FIG. 5A). They then tested whether LYVE-1 expressed on the cell surface of CHO cells could have an effect on FGF2 binding to low- or high-affinity binding sites and carried out binding experiments with radio labeled ¹²⁵I-FGF2 to CHO cells stably transfected with tmLYVE-1. As seen in FIG. 5B, CHO cells transfected with full-length LYVE-1 showed an increased ¹²⁵I-FGF2 binding to low affinity sites. Moreover, incubation of LYVE-1 transfected CHO cells in the presence of heparin resulted in a reduction of ¹²⁵I-FGF2 binding to low-affinity HSPGs (FIG. 5B).

To assess whether LYVE-1 could affect FGF2 binding to high-affinity tyrosine kinase FGFRs in CHO cells, they generated double transfected FGFR3-LYVE-1 CHO cells, using CHO cells stably transfected with FGFR3¹⁶, since FGFR3 is present on the cell surface of lymphatic endothelial cells³¹. Double-transfected clones were analyzed for LYVE-1 expression by qPCR and immunofluorescence. In contrast to FGF2 binding to low-affinity sites, LYVE-1 had no effect on ¹²⁵I-FGF2 binding to high-affinity sites (FIG. 5C).

Effect of LYVE-1 on FGF2-Induced Lymphangiogenesis In Vivo:

To analyze the effect of LYVE-1 on lymphangiogenesis in vivo the inventors applied a corneal lymphangiogenesis assay to quantitatively study the formation of new lymphatic vessels¹⁸. Earlier it has been shown that FGF2 implanted in the mouse cornea stimulated lymphangiogenesis^{7,19}. To analyze the effect of LYVE-1 on FGF2-induced lymphangiogenesis, pellets containing FGF2 alone or a combination of FGF2/LYVE-1 were implanted in corneas of BALB/c mice (Figure E). Implantation of pellets containing FGF2 alone resulted in the outgrowth of lymphatic vessels with a mean neovascularization area of 0.23±0.11 mm². On the contrary, in corneas with pellets containing FGF2/LYVE-1, the neovascularization area decreased around 48% (mean vascularization area of 0.12±0.07 mm², p=0.0008). This clearly indicates that LYVE-1 also inhibits FGF2 activity on lymphangiogenesis in vivo.

To ascertain the specificity, the effect of LYVE-1 on VEGF-C induced corneal lymphangiogenesis was investigated. VEGF-C-induced lymphangiogenesis and VEGF-C/LYVE-1-induced lymphangiogenesis did not differ significantly in the micropocket assay. This indicates that LYVE-1 does not inhibit the lymphangiogenic effect of VEGF-C in vivo.

FGF2-Dependent Regulation of LYVE-1 Expression:

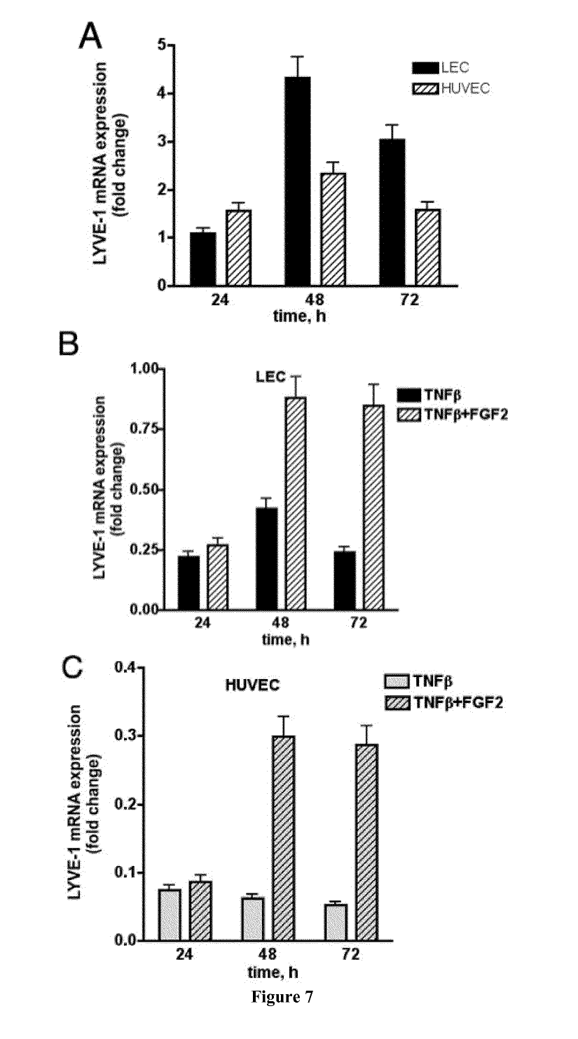

To further characterize the interaction between FGF2 and LYVE-1 in lymphatic cells, the inventors investigated whether FGF2 has an effect on LYVE-1 expression in LEC. Indeed, FGF2-stimulated LECs and HUVECs exhibited a maximum in LYVE-1 expression at 48 h (FIG. 7A). They next investigated whether FGF2 could reverse the TNFβ-dependent down-regulation of LYVE-1. LECs and HUVECs were stimulated with FGF2 for 24-72 h in the presence of TNFβ. As expected, FGF2 was able to reverse the TNFβ-dependent down-regulation of LYVE-1 in both cell types (FIG. 7 B, C). These results were confirmed by immunofluorescence labelling of LEC using anti-LYVE-1 antibodies. FGF2 increases the fluorescent signal for LYVE-1, while TNFβ significantly diminishes it. In the presence of FGF2, immunoreactivity for LYVE-1 is restored. They then examined whether FGF-2 is able to counteract the effect of TNFβ on LYVE-1 expression in an ex vivo assay. Ear explants from mice were exposed for 48 h to TNFβ (200 ng/ml) in the presence or absence of FGF2 (200 ng/ml). TNFβ clearly decreases the immunofluorescence signal for LYVE-1. This is no more observed when FGF2 is added together with TNFβ. Taken together, these results indicate that FGF2 positive regulates LYVE-1 expression in LEC and HUVEC and is able to counteract the inhibitory effect of TNFβ on LYVE-1 expression in vitro and ex vivo.

They then investigated whether the expression of endogenous LYVE-1 is downregulated in FGF2 knockout mice. Accumulating evidences indicate that LYVE-1 expression is not restricted to the lymphatic endothelium, but it is also expressed in the normal liver and spleen sinusoids, heart myocytes and lung macrophages. They isolated total RNA from liver, lung, spleen and heart of wild type male and female mice and examined the levels of LYVE-1 expression by qPCR. They found abundant LYVE-1 expression in wild type tissue of lung, heart and liver, and low LYVE-1 levels in spleen. Furthermore, they observed that the levels of endogenous LYVE-1 were not identical in male and female mice, especially in liver and spleen. Therefore they analyzed LYVE-1 expression levels in male and female FGF2 knockout mice separately, normalizing them to LYVE-1 level in wild type tissues (n=5). They found that LYVE-1 expression levels were decreased only in the liver and spleen of male mice, while female showed no difference in LYVE-1 expression in these organs. Thus FGF2 may positively regulate LYVE-1 expression in vivo in a tissue-specific manner. The data indicate that FGF2 is critical for LYVE-1 expression in liver and spleen sinusoids.

### DISCUSSION

LYVE-1 is a lymphatic endothelial cell marker that is also expressed in some other cell types including activated tissue macrophages, the sinusoidal endothelium of liver and spleen, heart myocytes as well as some tumors. However, its function is still not clearly elucidated. In the present specification, the inventors report that a member of the FGF family, FGF2 can interact with LYVE-1 and that this interaction impacts on some of the FGF2-mediated effects in the lymphatic endothelium. This is based on the following observation: (1) FGF2 is able to directly bind LYVE-1 in vitro as demonstrated using 4 different assays, (2) FGF2 interacts with LYVE-1 with a higher affinity than any other known LYVE-1 binding molecules including HA and PDGF-BB, (3) Glycosylation of LYVE-1 is important for the interaction with FGF2, (4) FGF2 is able to interact with LYVE-1 when LYVE-1 is overexpressed in CHO cells, (5) soluble LYVE-1 and knockdown of LYVE-1 in LEC have inhibitory effects on FGF2-induced migration and invasion, (6) LYVE-1 also inhibits lymphangiogenesis in vivo, (7) FGF2 can modulate LYVE-1's endogenous expression and reverse the effect of TNFβ and (8) FGF2 knockdown animals exhibit a down-regulation of LYVE-1 expression in some tissues.

They found that LYVE-1 binds FGF2 using AlphaScreen® technology. The interaction between FGF2 and LYVE-1 was concentration-dependent and a maximum signal was obtained at 500 nM FGF2 and 150 nM LYVE-1. These results were reinforced using solid phase binding assays with surface immobilized FGF2 and injected LYVE-1. Under these conditions, a significant binding was observed. The affinity as reflected by the calculated KD was of 100 nM and was 4-10 fold lower than those obtained for other LYVE-1 binding molecules tested including VEGFA, PDGF-BB, VEGFC, fibronectin and HA. This indicates a better affinity of LYVE-1 for FGF2 than for other binding molecules. We were also able to demonstrate that the FGF2/LYVE-1 complex occurred in solution using chemical cross-linking or co-immunoprecipitation experiments. Another observation of note is the fact that heparin and PDGF-BB were able to compete with the FGF2/LYVE-1 interaction. These findings indicate the existence of a probable overlapping binding site in LYVE-1 or FGF2 that also binds to heparin and PDGF-BB. Indeed, PDGF-BB has been reported to be an important growth factor contributing to lymphatic metastasis and bind LYVE-1 and our observations are in line with these data. These data suggest that LYVE-1 may be involved in the modulation of VEGF-C-independent lymphangiogenesis by directly interacting with critical lymphangiogenesis factors such as FGF2 or PDGF-BB. Moreover, it has also been reported that PDGF-BB directly interacts with FGF2 and thus PDGF-BB may also compete for LYVE-1 binding on FGF2. Heparin is known to be implicated in the dimerization of FGF2 and to modulate the interaction of FGF2 with FGF receptors and thus might compete for LYVE-1 binding on FGF2. However, the inverse may also be true.

In addition, they demonstrated that glycosylation of LYVE-1 is important for the interaction with FGF2. This may explain the completion of LYVE-1 by heparin, since the heparin-binding domain of FGF2 may be involved in the FGF2/LYVE-1 interaction. The exact nature of the interacting domains for FGF2 or LYVE1 is not known at the present moment.

They next investigated whether LYVE-1 was able to interact with FGF2 when overexpressed at the cell surface. They therefore transfected CHO cells with the pcDNA3.1/Hygro(+)vector encoding full-length LYVE-1. They clearly demonstrated a significant increase of FGF2 binding in CHO cells that overexpressed LYVE-1. This binding could also be competed with heparin, thus confirming the binding data obtained in vitro. When binding data were analyzed by distinguishing high affinity binding from low affinity binding, it appeared that only low affinity binding but not high affinity binding of FGF2 was increased. High affinity binding was tested specifically for FGFR3 since FGFR3 is overexpressed in lymphatic endothelial cells. Since LYVE-1 overexpression only modulates low-affinity binding to HSPGs but not high affinity binding, one must explain how LYVE-1 is implicated in the regulation of FGF2's activity. A substantial body of literature suggests that full activity of FGFs requires not only receptor interactions but also their internalization and that the latter may proceed via HSPG-dependent and—independent pathways. In this regard, LYVE-1 may participate in FGF2 internalization and subsequent FGF2 signalling inside of cell. LYVE-1 may also cooperate with some other not yet identified binding molecule that is important for FGF activity. For instance, transmembrane proteoglycan syndecan-4 may be a potential candidate. Indeed, it has been demonstrated that syndecan-4 is directly involved in FGF2 internalization in endocytic vesicles. Regarding these observations they can therefore speculate that LYVE-1 may help FGF2 in proteoglycan's binding such as syndecan-4. The present data are in agreement with this hypothesis since LYVE-1 increased low affinity binding of FGF2 in LYVE-1 transfected CHO cells, which is competed by heparin. Further studies are however needed to clarify whether LYVE-1 is involved in syndecan-4-dependent FGF2 interactions.

The inventors next sought to investigate whether LYVE-1 was able to alter some of FGF2's functions such as migration, invasion and proliferation. They observed that FGF2-stimulated migration, invasion and proliferation were inhibited in the presence of LYVE-1. They showed that FGF2-induced signalling was impaired by LYVE-1. These experiments were made by using both, LYVE-1 and siRNA for LYVE-1. Most importantly, sLYVE-1 is able to inhibit FGF2-induced lymphangiogenesis in vivo in a rat corneal lymphangiogenesis assay.

Taken together, the present results demonstrate that LYVE-1 is able to interfere with LEC function in vitro and in vivo. In agreement with these results it has been recently shown that LYVE-1 is involved in the disruption of VE-cadherin-mediated intercellular adhesion and opening of intercellular junctions in LEC monolayers. Furthermore, it has been demonstrated that the inhibition of FGF signalling is important for vascular integrity. Indeed, blockade of FGF activity in vitro and in vivo results in dissociation of the VE-cadherin/p120-catenin complex and disassembly of adherent and tight junctions, which progress to loss of endothelial cells and disintegration of the vasculature. Thus, the present results together with these observations point to an important interaction between FGFs and LYVE-1 in the lymphatic endothelium.

They also demonstrated that FGF2 not only interacts with LYVE-1 but can also induce its expression in LEC. More importantly, FGF2 is able to counteract the down-regulation of LYVE-1 by TNFβ in LEC. This is based on in vitro and ex vivo experimental evidences. It has been previously reported that LYVE-1 expression is down-regulated by TNFβ through internalization and degradation of receptors in lysosomes, coupled with a shutdown in gene expression. This is quite remarkable and may be of importance in chronic inflammation where TNF is abundantly produced. In some circumstances, FGF may be released by stroma cells and reverse the effect of TNF. In addition, it has been reported that FGF2 mediates a protective effect against TNFβ-mediated apoptosis in HUVEC. Recently, the presence of LYVE-1-negative gaps in the corneal lymphatic endothelium has been observed. The number of gaps in FGF2, but not VEGF A implanted corneas was significantly lower than in untreated corneas, which indicates a potential stimulatory effect of FGF2 on LYVE-1 expression. Another hypothesis is that in some tumors, FGF2 overexpression may influence the lymphatic endothelial cell phenotype. It has been shown that prox1, the homeobox-containing transcription factor expressed by LEC, is able to up-regulate FGFR3 receptor in LEC. Thus it is possible that FGF2 blocks external cues that negatively act on the lymphatic phenotype.

To reinforce the contention that FGF2 positively regulates LYVE-1 expression, we analysed whether FGF2 loss in FGF2−/− mice led to a decrease of LYVE-1 expression in some of their organs. Indeed, we show a decrease in LYVE-1 expression in the liver and spleen of FGF2−/− mice. These organs are responsible for the uptake and degradation of high molecular weight HA11.

Thus, FGF2 and LYVE-1 are possibly integrated in a regulatory loop, in which FGF2 activates LYVE-1 expression, which in turn, limits FGF2 activity by directly interacting with the growth factor.

In conclusion, the inventors show for the first time that LYVE-1 is able to interact with one of the fibroblast growth factor members; namely FGF2. This interaction appears stronger than any of other known LYVE-1 binding molecules. Furthermore, FGF2/LYVE-1 interactions may be of importance for some of the FGF2's related effects in the vasculature and for in vivo lymphangiogenesis. Finally, FGF2 prevents the downregulation of LYVE-1-induced by pro-inflammatory cytokines such as TNFβ^{˜}.

### REFERENCES

Throughout this application, various references describe the state of the art to which this invention pertains. The disclosures of these references are hereby incorporated by reference into the present disclosure.
1. Baldwin M, Stacker S, Achen M. Molecular control of lymphangiogenesis. BioEssays. 2002; 24:1030-1040.
2. Cao Y. Angiogenesis and lymphangiogenesis in common diseases. Editorial. Curr Mol Med. 2009; 9(8):928.
3. Alitalo K, Tammela T, Petrova T V. Lymphangiogenesis in development and human disease. Nature. 2005; 15:438(7070):946-953.
4. Cao R, Eriksson A, Kubo H, Alitalo K, Cao Y, Thyberg J. Comparative evaluation of FGF-2-, VEGF-A-, and VEGF-C-induced angiogenesis, lymphangiogenesis, vascular fenestrations, and permeability. Circ Res. 2004; 94(5):664-70.
5. Cao Y, Zhong W. Tumor-derived lymphangiogenic factors and lymphatic metastasis. Biomed Pharmacother. 2007; 61(9):534-9.
6. Cao Y. Opinion: emerging mechanisms of tumour lymphangiogenesis and lymphatic metastasis. Nat Rev Cancer. 2005; 5(9):735-43.
7. Chang L K, Garcia-Cardena G, Farnebo F, et al. Dose-dependent response of FGF2 for lymphangiogenesis. Proc Natl Acad Sci USA. 2004; 101:11658-11663.
8. Shin J W, Min M, Larrieu-Lahargue F, et al. Prox1 promotes lineage-specific expression of fibroblast growth factor (FGF) receptor-3 in lymphatic endothelium: a role for FGF signaling in lymphangiogenesis. Mol Biol Cell. 2006; 17(2):576-584.
9. Cao R, Ji H, Feng N, et al. Collaborative interplay between FGF-2 and VEGF-C promotes lymphangiogenesis and metastasis. Proc Natl Acad Sci USA. 2012; 109(39):15894-9.
10. Kubo H, Cao R, Brakenhielm E, Mäkinen T, Cao Y, Alitalo K. Blockade of vascular endothelial growth factor receptor-3 signaling inhibits fibroblast growth factor-2-induced lymphangiogenesis in mouse cornea. Proc Natl Acad Sci USA. 2002; 99(13):8868-73.
11. Roghani M, Moscatelli D: Basic fibroblast growth factor is internalized through both receptor-mediated and heparan sulfatemediated mechanisms. J Biol Chem. 1992; 267(31):22156-62
12. Perollet C, Han Z C, Savona C, Caen J P, Bikfalvi A. Platelet factor 4 modulates fibroblast growth factor 2 (FGF2) activity and inhibits FGF2 dimerization. Blood. 1998; 91(9):3289-3299.
13. Aidoudi S, Bujakowska K, Kieffer N, Bikfalvi A. The CXC-chemokine CXCL4 interacts with integrins implicated in angiogenesis. PLoS One. 2008; 3(7):e2657.
14. A Practical Guide to working with AlphaScreen. PerkinElmer Life and Analytical Sciences; 2004.
15. Mohammadi M, Dikic I, Sorokin A, Burgess W H, Jaye M, Schlessinger J. Identification of six novel autophosphorylation sites on fibroblast growth factor receptor 1 and elucidation of their importance in receptor activation and signal transduction. Mol Cell Biol. 1996; 16(3):977-89.
16. Rusnati M, Dell'Era P, Urbinati C, et al. A distinct basic fibroblast growth factor (FGF2)/FGF receptor interaction distinguishes urokinase-type plasminogen activator induction from mitogenicity in endothelial cells. Mol Biol Cell. 1996; 7(3):369-381.
17. Bikfalvi A. Angiogenesis: health and disease. Ann Oncol. 2006; 17 (Suppl 10):65-70.
18. Cao R, Lim S, Ji H, et al. Mouse corneal lymphangiogenesis model. Nat Protoc. 2011; 6(6):817-26.
19. Regenfuss B, Onderka J, Bock F, Hos D, Maruyama K, Cursiefen C. Genetic heterogeneity of lymphangiogenesis in different mouse strains. Am J Pathol. 2010, 177(1):501-10.
20. Johnson L A, Prevo R, Clasper S, Jackson D G. Inflammation-induced uptake and degradation of the lymphatic endothelial hyaluronan receptor LYVE-1. J Biol Chem. 2007; 282(46):33671-33680.
21. Patry V, Bugler B, Amalric F, Promé J C, Prats H. Purification and characterization of the 210-amino acid recombinant basic fibroblast growth factor form (FGF-2). FEBS Lett. 1994 25; 349(1):23-8.
22. Rogers M S, Birsner A E, D'Amato R J. The mouse cornea micropocket angiogenesis assay. Nat Protoc. 2007; 2(10):2545-50.
23. Bock F, Onderka J, Hos D, Horn F, Martus P, Cursiefen C. Improved semiautomatic method for morphometry of angiogenesis and lymphangiogenesis in corneal flatmounts. Exp Eye Res. 2008; 87(5):462-70.

1. A method for preventing or treating a pathological condition associated with lymphangiogenesis and/or for preventing tumor metastasis in a subject in need thereof comprising administering to said subject a therapeutically effective amount of a Lymphatic Vessel Endothelial Hyaluronan Receptor-1 (LYVE-1) antagonist.

2. (canceled)

3. The method of claim 1, wherein the LYVE-1 antagonist is an inhibitor of the interaction between LYVE-1 and Fibroblast Growth factor-2 (FGF-2).

4. The method of claim 1, wherein said antagonist is a LYVE-1 receptor polypeptide.

5. The method of claim 1, wherein said antagonist is an anti-LYVE-1 neutralizing antibody or aptamer.

6. The method of claim 1, wherein said antagonist is an inhibitor of LYVE-1 gene expression.

7. The method of claim 6, wherein said inhibitor of LYVE-1 gene expression is a small inhibitory RNA (siRNA), a ribozyme, or an antisense oligonucleotide.

8. The method of claim 1, wherein the pathological condition associated with lymphangiogenesis is selected from the group consisting of cancer, an eye disease and an inflammatory disease.

9. A pharmaceutical composition comprising a LYVE-1 antagonist selected from the group consisting of a LYVE-1 receptor polypeptide, an anti-LYVE-1 neutralizing antibody or aptamer and an inhibitor of LYVE-1 gene expression and a pharmaceutically acceptable carrier.

10. The pharmaceutical composition according to claim 9 further comprising an additional therapeutic agent.

11. A kit-of-part composition comprising at least a LYVE-1 antagonist selected from the group consisting of a LYVE-1 receptor polypeptide, an anti-LYVE-1 neutralizing antibody or aptamer and an inhibitor of LYVE-1 gene expression and an additional therapeutic agent.

12. The pharmaceutical composition according to claim 10, wherein said additional therapeutic agent is a VEGFR-3 antagonist.

13. (canceled)

14. A method for screening a LYVE-1 antagonist comprising the steps of:
a) providing a plurality of cells expressing LYVE-1 on their surface;
b) incubating said cells with a candidate compound;
c) determining whether said candidate compound binds to and inhibits LYVE-1; and
d) selecting the candidate compound that binds to and inhibits LYVE-1.

15. A method for screening a LYVE-1 antagonist comprising the steps of:
a) determining the ability of a candidate compound to inhibit the interaction between a LYVE-1 polypeptide and a FGF2 polypeptide, and
b) selecting positively the candidate compound that inhibits said interaction.

16. The kit-of-part composition of claim 11, wherein said additional therapeutic agent is a VEGFR-3 antagonist.

17. The method of claim 8, wherein said eye disease is selected from the group consisting of macular degeneration and diabetic retinopathy.

18. The method of claim 8, wherein said inflammatory disease is selected from the group consisting of rheumatoid arthritis, diabetes and psoriasis.


SEQUENCE LISTING (1 sequence)

SEQ ID NO: 1 - protein, 322 aa, Homo sapiens
MARCFSLVLLLTSIWTTRLLVQGSLRAEELSIQVSCRIMGITLVSKKANQQLNFTEAKEACRLLGLSLAGKDQVETALKASFETCSYGWVGDGFVVISRISPNPKCGKNGVGVLIWKVPVSRQFAAYCYNSSDTWTNSCIPEIITTKDPIFNTQTATQTTEFIVSDSTYSVASPYSTIPAPTTTPPAPASTSIPRRKKLICVTEVFMETSTMSTETEPFVENKAAFKNEAAGFGGVPTALLVLALLFFGAAAGLGFCYVKRYVKAFPFTNKNQQKEMIETKVVKEEKANDSNPNEESKKTDKNPEESKSPSKTTVRCLEAEV